(19) United States
(12) Patent Application Publication
(10) Pub. No.: US 2021/0223266 A1
(43) Pub. Date: Jul. 22, 2021

(54) METHODOLOGIES AND METHODS FOR MEASURING HIGHER MOLECULAR WEIGHT TRANSTHYRETIN OR EQUIVALENTS AS A CLINICAL BIOMARKER

(71) Applicant: TTR Therapeutics, New York, NY (US)

(72) Inventor: Gobind Singh, New York, NY (US)

(21) Appl. No.: 16/798,033

(22) Filed: Feb. 21, 2020

Related U.S. Application Data
(60) Provisional application No. 62/808,521, filed on Feb. 21, 2019.

(51) Int. Cl. G01N 33/68; G01N 33/563

(52) U.S. Cl. CPC G01N 33/6893; G01N 2800/52; G01N 33/563

(57) ABSTRACT

This invention provides methodologies and methods for measuring concentrations of higher molecular weight (HMW) transthyretin (TTR) species, or functional equivalents, in a biological sample that are useful as a clinically applicable biomarker (diagnostic, prognostic, predictive, treatment response, safety) for multiple diseases and conditions. Our discovery is immediately applicable and generally available for the general practitioner for use in diseases where currently few to no biomarkers available like light-chain amyloidosis (AL), a B-cell/plasma cell cancer/dyscrasia that is also associated with multiple myeloma, monoclonal gammopathy of undetermined significance (MUGS), B-cell lymphomas, and Waldenstrom macroglobulinemia. Methodologies and methods are disclosed.

### CROSS-REFERENCE TO RELATED APPLICATION

The present application claims the benefit of U.S. Provisional Patent Application No. 62/808,521, filed Feb. 21, 2019, whose disclosure is hereby incorporated by reference in its entirety into the present disclosure.

### FIELD OF THE INVENTION

This invention provides methodologies and methods for measuring concentrations of higher molecular weight (HMW) transthyretin (TTR) species, or functional equivalents, in a biological sample that are clinically applicable as a biomarker (diagnostic, prognostic, predictive, treatment response, safety) for multiple diseases and conditions. In the context of light-chain amyloidosis (AL) and associated cancer conditions, these methods enable a new biomarker to have practical clinical utility that can be used for ameliorating, treating, or preventing poor clinical outcomes in a human subject receiving immunotherapy or immunomodulatory agents.

### DESCRIPTION OF RELATED ART

Amyloid (amyloid-beta, Aβ, beta-amyloid, or β-amyloid) is a proteinaceous material that is the byproduct of proteins that have misfolded. Aβ is pathologic to human tissues. Many proteins have been identified as amyloid “precursor proteins”, including transthyretin (TTR) and immunoglobulin-derived light-chains (LC), which are two sources of amyloid that are known to affect cardiac function with serious clinical sequelae, including death.

TTR monomers can oligomerize to form dimers and tetramers. Various models have been proposed that discuss how these different TTR isoforms exist in equilibrium, including the breakdown of TTR tetramer into TTR monomers, which are the species thought to be most prone to misfolding and forming beta-rich amyloid fibrils (Aβ). TTR mediated amyloid formation can occur with non-mutated TTR (wild-type) or mutated TTR. Certain mutations have been discovered to destabilize the tetrameric form thereby accelerating breakdown and amyloid formation and associated with worse clinical outcomes. In contrast, some TTR mutations have been found to promote tetramer stabilization (i.e, T119M) and are associated with clinical benefit, including protection against amyloid mediated diseases, many of which cardiovascular.

Many assays are available for quantitating TTR levels. Of those that are immunoassays, there are many anti-TTR antibodies that each can affect quantitation of TTR levels differently depending on assay conditions and antibody binding specificity for each TTR quaternary isoform (tetramer, dimer, monomer, etc). Prior medical inventions that have discussed TTR focused on different classes of agents that modulated transthyretin (TTR) levels for treatment purposes. However little or no methods were specified for quantitating TTR level changes in the clinical arena. In-part, this is because TTR levels historically, as measured in the clinic, are thought to be variable, unreliable, and/or non-specific (“Transthyretin [Prealbumin] and the Ambiguous Nature of Malnutrition,” J. Hosp. Med. 2019 April; 14(4):257-258; “Transthyretin for the routine assessment of malnutrition: A clinical dilemma highlighted by an international survey of experts in the field,” Clin Nutr. 2018 December; 37[6 Pt A]:2226-2229; “Things We Do for No Reason: Prealbumin Testing to Diagnose Malnutrition in the Hospitalized Patient,” J Hosp Med. 2019 April; 14[4]:239-241). As the function of tetrameric TTR protein remains mostly elusive beyond its role as a transporter protein, it is challenging to design functional assays that may correlate function to levels.

The problem at hand exists with the following hypothetical example; clinical reports in disease Y found TTR levels were increased relative to healthy controls. This is in contrast to a separate study also in disease Y that instead reported decreased TTR levels relative to healthy controls. Conflicting reports like this are the cause for clinicians not having confidence in TTR level assays and highlight the non-obviousness for how to reliably measure clinically applicable TTR levels. In-fact, limited understanding has prevented its application in medicine such that agents that modulate TTR levels are currently FDA approved for patients with TTR mediated diseases (i.e., ATTR), yet without the recommendation of ever measuring TTR levels before or after treatment, including gauging treatment response. While new tools like bone scintigraphy imaging have improved ATTR-CM diagnosis-rates, there are still cardiac involving ATTR populations (i.e., F64L) that bone scintigraphy has proven ineffective (“Low Sensitivity of Bone Scintigraphy in Detecting Phe64Leu Mutation-Related Transthyretin Cardiac Amyloidosis,” https://doi.org/10.1016/j.jcmg.2019.10.015) or non-specific (AL). Also, while there are novel imaging agents (Blood, Volume 134, Issue Supplement_1, Nov. 13, 2019) and assays for misfolded TTR (WO 2016/120811 A1) being developed and not commercially available, our discovery provides methods that include some which are already commercially available and can unlock the biomarker application of higher-molecular weight (HMW) TTR levels across diseases and symptoms linked to amyloidosis. Examples that would immediately benefit from this invention include immunoglobulin light-chain amyloidosis (AL) and associated cancers along with transthyretin amyloidosis (ATTR) and related conditions.

### BACKGROUND OF THE INVENTION

Transthyretin (TTR, TTY), also known as prealbumin (PAB, PA) or thyroxine-binding prealbumin (TBPA), is thought to have been first identified in 1955 in the cerebrospinal fluid (CSF) when it was called “component X” (“A study of normal human cerebrospinal fluid by the immuno-electrophoretic method,” Nature. 1955 Nov. 19; 176(4490):976.). TTR monomers, β-sheet-rich proteins that can oligomerize, are normally synthesized at multiple locations in the human body including the liver, choroid plexus, pancreas, retina, stomach, spleen, heart, and the placenta. Largest TTR concentrations are found in the serum and cerebrospinal fluid (CSF), with TTR in the CSF being the second most abundant protein and the primary carrier of thyroid hormone. Other bodily fluids where TTR has been detected include urine, vitreous fluid, and saliva.

TTR tetramer is a 55 kilodalton (kDa) protein composed of four TTR monomers that are each 13.8 kDa. Its name has undergone multiple changes alongside the evolution of the understanding of TTR tetramer function. The current name, trans-thy-retie, reflects TTRtetramer function as a transporter protein of both retinol (vitamin A), through association with retinol-binding protein (RBP), and thyroxine (T4), a thyroid hormone. Other functions of tetrameric TTR remain poorly understood. However, it's worth noting isolated reports have discussed: (1) protease function with substrates like ApoA-I, neuropeptide Y (NPY), and beta-amyloid (Aβ), (2) detoxifier function including Aβ, and (3) protein-protein interactions including lipoprotein receptor-related protein 1 (LRP1) or glucose-regulated proteins (GRPs). As TTR tetramer function is better understood in the future, assays for measuring tetramer function could also be used instead of HMW TTR levels, herein referred to as functional equivalents.

Different assays are available for detecting and measuring TTR levels in a biological sample. Whether directly or indirectly measuring TTR concentrations, many of these assays do not distinguish tetramer quaternary isoforms from smaller TTR forms. For example, if a biological sample was primarily composed of TTR monomers, an assay that measured TTR tetramer levels would output low TTR levels. In contrast, another assay that measured TTR monomer levels in this same biological sample would instead output high TTR levels. Depending on the assay used, high or low TTR levels could be reported for the same biological sample. This has been problematic clinically when trying to develop TTR levels as a biomarker, including attempts to correlate high or low TTR levels with a specific disease or condition. For example, there are conflicting reports on whether pancreatic ductal adenocarcinoma (PDAC) patients' vs healthy controls had higher or lower TTR levels. One publication (“Identification and verification of transthyretin as a potential biomarker for pancreatic ductal adenocarcinoma,” J Cancer Res Clin Oncol. 2013 July; 139(7):1117-27. doi: 10.1007/s00432-013-1422-4. Epub 2013 Apr. 2), which used ELISA and PCR methods, reported significantly higher TTR levels in PDAC in contrast to another study (“Improved Pancreatic Adenocarcinoma Diagnosis in Jaundiced and Non-Jaundiced Pancreatic Adenocarcinoma Patients through the Combination of Routine Clinical Markers Associated to Pancreatic Adenocarcinoma Pathophysiology,” https://doi .org/10.1371/journal.pone.0147214), which used nephelometry, and instead reported significantly lower TTR levels in PDAC. With conflicting reports like this regarding TTR levels, it's not surprising TTR levels have not been successfully developed as a biomarker in the U.S and are not recommended for patients even if on FDA-approved TTR modulating agents for TTR-mediated diseases (i.e, tafamidis [VyndaquelNyndamax] for ATTR-cardiomyopathy [ATTR-CM]; Patisiran [Onpattro] and inotersen [Tegsedi] for ATTR-polyneuropathy [ATTR-PN]).

Probably the most common reason for measuring TTR levels is to assess nutritional status, with decreasing TTR levels linked to worsening malnutrition. However, the story may turn out to be more complicated as recently published reports showed disease-related malnutrition is distinct from disease-related malnutrition plus inflammation, with the latter group linked to significantly greater weight loss and deterioration of endothelial dysfunction (“Malnutrition impairs mitochondrial function and leukocyte activation,” Nutrition Journal volume 18, Article number: 89 (2019)). In AL patients, while nutritional status is an independent prognostic factor, recent studies showed decreased HMW TTR levels were significantly linked to decreased functioning of numerous physical and mental components (“Nutritional status independently affects quality of life of patients with systemic immunoglobulin light-chain (AL) amyloidosis,” Ann Hematol. 2012 March; 91(3):399-406. doi: 10.10071s00277-011-1309-x. Epub 2011 Aug. 9). While some may attribute lower TTR levels as non-specific behaving like a negative acute phase reactant should, there are reports which have clearly shown TTR levels are not decreased in all inflammatory diseases, but in-fact elevated in some (“Altered cerebrospinal fluid index of prealbumin, fibrinogen, and haptoglobin in patients with Guillain-Barré syndrome and chronic inflammatory demyelinating polyneuropathy,” Acta Neurol Scand. 2012 February; 125(2):129-35. doi: 10.1111/j.1600-0404.2011.01511.x. Epub 2011 Mar. 24. discussed further below).

### SUMMARY OF THE INVENTION

The invention is important because it provides methodologies and methods for measuring HMW TTR levels in a biological sample for use as a clinical biomarker. In a time when TTR-mediated diseases are treated with FDA approved drugs that modulate TTR levels irrespective of baseline TTR levels, this patent discovery is timely and provides the methodology and methods for measuring HMW TTR levels that are clinically meaningful. In other aspects, this invention features specific methods for measuring HMW TTR levels including methods that are (1) available to health-care providers, (2) cost-effective for patients, and (3) have already demonstrated their use as a clinically significant biomarker. As used herein, the term “high molecular weight (HMW) transthyretin (TTR)” species or “HMW TTR” species references TTR tetramers or functional equivalents, or a mixture of TTR HMW species where the predominant species are TTR tetramers or the functional equivalent thereof The invention provides methodologies and methods that enable HMW TTR levels to be clinically useful for multiple applications.

TTR is known to be a highly conserved protein across species. In humans, there are multiple sites of synthesis including the liver and choroid plexus of the brain (primary) in addition to the retina, pancreas, kidneys, and neurons. TTR monomers are beta-sheet rich proteins, each ˜14 kilodaltons (kDa) that noncovalently combine to form dimers and tetramers, with the latter synthesized as a non-glycosylated tetramer with a molecular mass of ˜55 kDa. When bound to holo-retinol-binding protein (RBP; ˜21 kDa), this results in a combined MW of ˜76 kDa. Post-translational modifications can also occur (i.e., sulfonation, cysteinylation, glutathionylation, etc). Physiologically, different TTR isoforms (i.e., monomers, dimers, tetramers, truncated) exist in equilibrium with each other, with the tetramer structure uniquely creating two binding pockets for thyroid hormone. TTR monomers on the other hand are thought to be most prone to protein misfolding resulting in toxic byproducts that deposit and cause damage to human tissues/organs. Currently, TTR tetramer is not thought to be the main transporter of thyroid hormone in the plasma/serum. This is in contrast to the CSF where TTR tetramer is thought to be the main transporter of thyroid hormone to the brain.

HMW TTR levels have been shown to have at least similar if not greater prognostic value than many of the biomarkers currently recommended in U.S guidelines for different diseases/conditions including: (1) accessing liver function pre-transplantation (i.e., Child Pugh Score), (2) accessing renal damage in AL amyloidosis (i.e., revised mayo clinic score used NT-proBNP, cardiac troponin T [CTnT], difference between involved and uninvolved free light chain concentration [dFLC]), or (3) staging patients with ATTR-CM disease. The methodologies and methods provided herein enable TTR levels to be used as a clinical biomarker with broad applications.

Since the 1970s in an article published in The Lancet, TTR levels have been proposed as an indicator of protein-energy status (PEM), with subsequent reports showing TTR levels as a biomarker across an array of diseases (see Ingenblee, Y et al., “Measurement of prealbumin as index of protein-calorie malnutrition, Lancet 1972). Use of TTR assays in the U.S gained momentum such that low levels permitted a clinical diagnosis of PEM and treatment with reimbursement from third-party payers like Medicare and Medicaid (see Johnson, A. Low levels of plasma proteins. Clin Chem Lab Med 1999). As an example showing TTR levels as a biomarker, a study randomized 501 geriatrics (with different diseases) for 26 weeks to receive either nutritional supplementation on top of standard hospital diet or hospital diet alone (see (Larsson, J et. al Clinical Nutrition 1990)). Important takeaways from this study included amongst those with baseline poor nutrition (before randomization), greater death-rates (37% vs 29%) were observed in the non-supplemented vs supplemented, which correlated with lower week one TTR levels (22 vs 19 mg/dL). A similar trend for greater deaths (19% vs 9%) that correlated with lower TTR levels was also seen in the adequate baseline nutrition group. Despite the promising potential of TTR levels as shown here, a 2018 international survey from experts in the malnutrition field reported that even though TTR levels exist in treatment guidelines of other countries (e.g., UK, France, Italy), no formal guidelines in the U.S have implemented TTR levels, in part due to the perception of it being a “poor marker of nutritional status (inflammation)” (see Delliere, S, et al Transthyretin for the routine assessment of malnutrition, Clinical Nutrition 2018). This is in-line with the International Federation of Clinical Chemistry and Laboratory Medicine report, which found that TTR concentrations may vary “as much as 50% or more above or below the true value.”

Our discovery hopes to clarify potential misconceptions by speaking directly to the above mentioned discrepancies, including identification of specific protocols that can TTR levels so that they are clinically relevant. Also, given 2019 was the year when the first drug was approved to treat a fatal heart disease directly linked to TTR breakdown (ATTR-CM), we believe our discovery is time-sensitive and provides practical clinical algorithms backed up by clinical data that clinicians can use for this disease and more, as further discussed.

Multiple assays exist for measuring TTR levels in a patient's biological sample, e.g., bodily fluid and/or tissue samples. However, many of these tests do not indicate which isoform is being measured nor do they distinguish between relative concentrations of each TTR isoform. Due to different methods being used to measure TTR levels, this has resulted in wide variations of TTR levels being reported, even conflicting at times despite being from the same patient population. For example, one publication used an ELISA kit from USCN Life Science and showed significantly higher TTR levels were present in serum from 40 pancreatic ductal adenocarcinoma (PDAC) patients compared 40 normal controls (median TTR level of 29.07 vs 24.95 mg/dL; p=0.009) (see Chen, J et al Identification and verification of TTR . . . J Cancer Res Clin Oncol 2013). This is in contrast to another study that used nephelometry to measure TTR levels and reported significantly lower TTR levels in PDAC patients compared to healthy controls (16.5 vs 25.5 mg/dL; “Improved Pancreatic Adenocarcinoma Diagnosis in Jaundiced and Non-Jaundiced Pancreatic Adenocarcinoma Patients through the Combination of Routine Clinical Markers Associated to Pancreatic Adenocarcinoma Pathophysiology,” PLoS One. 2016 Jan. 25; 11(1):e0147214. doi: 10.1371/journal.pone.0147214. eCollection 2016). This study used a different manufacturer's ELISA kit to measure TTR (Immundiagnostik AG) (see Ehmann, Michael. Identification of potential markers for the detection of pancreatic cancer through comparative serum protein expression profiling. Pancreas 2007). Yet another study of 20 PDAC patients reported PDAC patients had relatively lower TTR levels when compared to chronic pancreatitis. This study used western blotting and Dako's anti-TTR antibody. While it remains to be verified whether differences in antibody selection, method used, speed of sera centrifugation (4,000 g vs 2,000 g), or another variable are responsible for these varying TTR levels, it is clear that other unknown variables have not been accounted for and have resulted in confusion in the field resulting in clinicians have little confidence in TTR levels. More importantly, it explains why patients with fatal diseases have not been able to benefit from this biomarker to date.

The methodologies and methods provided herein help overcome the obstacles associated with current TTR detection methods by specifically identifying which methods measure HMW TTR levels such that they are clinically valuable and consistent. Herein we provide examples where HMW TTR species and/or functional equivalents have already demonstrated to be significantly useful on multiple functional clinical outcomes in numerous diseases and conditions. For specific diseases, we employ an algorithm that utilizes HMW TTR levels to help in disease screening, predict disease progression, or gauge treatment responsiveness whereas generally any drop in HMW TTR levels below 10 mg/dL should be taken seriously.

Methods of detecting high molecular weight (HMW) TTR species, including TTR tetramers, can be detected using any of a variety of known assays and/or reagents. Suitable methods for detecting HMW TTR species, including TTR tetramers, include turbidity-based or nephelometry-based methods. We point out these types of methods are commercially available from multiple manufacturers, allowing adoption into clinical practice immediately. Other suitable methods (albeit potentially less accessible and/or practical for widespread use like single radial immunodiffusion [SRID] assays) for detecting and quantifying HMW TTR species, including TTR tetramers, are described in Rappley et al., Biochem. (2014) 53(12): 1993-2006.

While the examples provided herein use TTR tetramers or combinations in which TTR tetramers are the predominant TTR species, the skilled artisan will appreciate that the following methods can also be used to detect any high molecular weight TTR species, including, by way of non-limiting example, TTR trimers, TTR tetramers, combinations of TTR trimers and TTR tetramers, and any mutants, truncates, or other variants thereof.

This invention provides methodology and methods for diagnosing a subject at risk of a poor clinical outcome by higher molecular weight (HMW) transthyretin (TTR) levels and/or their functional equivalents. Our invention is distinct in that others may have measured all TTR isoforms but did not report relative isoform concentrations, measured percentage of TTR stabilization ex-vivo relative to baseline levels after exposure to various conditions or agents, measured percentage of TTR reduction in-vivo but did not report absolute HMW TTR levels. In some embodiments, the method includes the steps of (1) providing a biological sample from the subject, and (2) detecting the level of higher molecular weight (HMW) transthyretin (TTR) species or a functional equivalent thereof present in the biological sample, wherein levels of HMW TTR species in the biological sample that fall below a critical threshold indicates an increased risk of a poor clinical outcome. Poor clinical outcomes are wide-spanning and are not limited to only heart failure or death.

For certain indications relating to prognosis of a patient's risk for certain clinical outcomes, aka poor clinical outcomes of a subject, the steps of (a) providing a biological sample from the subject, and (b) detecting a level of HMW TTR species or a functional equivalent thereof present in the biological sample, wherein a detected level of HMW TTR species in the biological sample below a critical threshold indicates an increased risk of poor clinical outcome(s) in a subject.

The methodology also includes methods of predicting risk of heart failure or death in a subject, the method comprising: (a) obtaining a biological sample from the subject, and (b) detecting a level of high molecular weight (HMW) transthyretin (TTR) species or a functional equivalent thereof present in the biological sample, wherein the detected level of HMW TTR species in the biological sample is used to stratify the patient into a category of risk for a poor clinical outcome, such as, for example, heart failure or death, based on HMW TTR levels.

In some embodiments, any of these methods further comprises step (c) comparing the detected level of HMW TTR species or functional equivalent thereof in the biological sample to either (1) a pre-treated HMW TTR levels from the same patient or (2) a control sample of HMW TTR species in a control sample from a control subject, wherein a decrease in HMW TTR levels in the biological sample as compared to the control level of HMW TTR species indicates an increased risk of a poor clinical outcome, including heart failure or death.

In some embodiments of any of these methods, the subject is at risk for or suffering from a TTR-related or TTR-associated disease or disorder.

The methodology also includes methods of evaluating responsiveness to a treatment regimen in a subject, the method comprising detecting a level of HMW transthyretin (TTR) species or a functional equivalent thereof at multiple time-points during the treatment regimen, and comparing the detected levels of HMW TTR species, wherein an increase in detected level of HMW TTR species indicates that the subject is responding to the treatment, and wherein a decrease in the detected level of HMW TTR species indicates that the subject is not or no longer responding to the treatment regimen.

The methodology also provides methods of screening HMW TTR species or a functional equivalents thereof or a combination of TTR species where TTR tetramers are the predominant species as a biomarker for a disease or disorder, with the method comprising: (a) providing biological samples from patients at risk or suffering from the disease or disorder; (b) detecting the level of HMW TTR species in each biological sample from a patient or in the combination of HMW TTR species in each biological sample from a patient; and (c) correlating the detected level of HMW TTR species in each biological sample from each patient or in the combination of TTR species in each biological sample from each patient with disease status.

The methodology also provides methods of selecting a treatment regimen by stratifying patient population based on transthyretin (TTR) tetramer levels or a functional equivalent thereof in a biological sample from each patient or TTR tetramer levels in a combination of TTR species in a biological sample from each patient.

In some embodiments of any of these methods, the higher molecular weight TTR species is TTR tetramers or a functional equivalent thereof or a mixture of TTR species where the predominant species is TTR tetramers.

In some embodiments of any of these methods, the subject is at risk of or suffering from a disease that is not currently linked to TTR or LC. In some embodiments of any of these methods, the subject appears to be healthy. In some embodiments of any of these methods, the subject is elderly.

In some embodiments of any of these methods, the subject has or is at risk for transthyretin-mediated amyloidosis (ATTR) or light-chain amyloidosis (AL). In some embodiments of any of these methods, the subject has or is at risk for ATTR-cardiomyopathy or ATTR-polyneuropathy. In some embodiments of any of these methods, the subject has or is at risk if protein malnutrition, stroke, coronary disease, heart failure, Alzheimer's disease, vitreous floaters, an infectious disease such as tuberculosis, glaucoma, ocular myasthenia gravis, cerebral amyloid angiopathy, beta-amyloid-related angiitis, atrial fibrillation, or leptomeningeal amyloidosis and variants like oculoleptomeningeal amyloidosis. In some embodiments of any of these methods, the subject is a liver transplant recipient. In some embodiments of any of these methods, the subject is on hemodialysis or peritoneal dialysis.

Our discovery is clinically translatable immediately, including for diseases with few to none biomarkers and few to no approved therapies, including ATTR-cardiomyopathy (ATTR-CM) or light-chain amyloidosis (AL), the latter being a B-cell cancer and proteinopathy (“Stabilization of amyloidogenic immunoglobulin light chains by small molecules,” PNAS Apr. 23, 2019 116 (17) 8360-8369) that is associated with multiple cancer-types like multiple myeloma (MM), B-cell lymphomas, Waldenstrom macroglobulinemia, and monoclonal gammopathy of undetermined significance (MUGS). Methodologies and methods are disclosed.

In some embodiments of any of these methods, the biological sample is a tissue sample. In some embodiments of any of these methods, the biological sample is a bodily fluid. In some embodiments of any of these methods, the bodily fluid is blood or a blood product such as serum or plasma, urine, cerebrospinal fluid (CSF), vitreous fluid, or a combination thereof.

In some embodiments of any of these methods, the bodily fluid is blood or a blood product such as serum or plasma, and wherein the critical threshold level for the lower level of normal limit is 18-20 mg/dL.

In some embodiments of any of these methods, the patient has been identified as having a low level of HMW TTR species.

In some embodiments of any of these methods, step (b) comprises using an antibody or other affinity ligand that specifically binds to TTR tetramer or another HMW TTR species and a methodology that preferentially detects HMW TTR species. In some embodiments of any of these methods, wherein step (b) comprises using turbidity-based or nephelometry-based methods.

In some embodiments of any of these methods, step (a) further comprises the step of isolating TTR tetramer or HMW variants from other TTR isoforms in the biological sample.

The invention in some embodiments also provides methods for measuring HMW TTR levels as it relates to monitoring treatment efficacy for AL and associated cancer conditions, delaying the progression of a transthyretin (TTR)-related and associated diseases, or preventing heart failure in a subject at risk for or suffering from a TTR-related disorder, the method comprising: (a) providing a biological sample from the subject, (b) detecting a level of transthyretin (TTR) tetramer or a functional equivalent or HMW TTR species present in the biological sample, and (c) administering a therapeutically effective amount of a therapeutic agent to increase or stabilize the level of HMW TTR species in the subject when there is a detected level of HMW TTR species below a critical threshold of normal in the biological sample.

The methodology also provides methods of monitoring the efficacy of a therapeutic regimen in a person at risk for or suffering from transthyretin (TTR) related disorder, the method comprising: (a) providing a first biological sample from the subject at a first-time point, (b) detecting a first level of HMW transthyretin (TTR) or a functional equivalent in the first biological sample, (c) providing a second biological sample from the subject at a second-time point, (d) detecting a second level of TTR tetramer or functional equivalent thereof or HMW TTR species present in the second biological sample, and (e) comparing the first detected level of TTR tetramer or functional equivalent thereof or HMW TTR species to the second detected level of TTR tetramer or functional equivalent thereof or HMW TTR species, wherein a decrease in the second detected level of TTR tetramer or functional equivalent thereof or HMW TTR species as compared to the first detected level of TTR tetramer or functional equivalent thereof or HMW TTR species indicates that the therapeutic regimen is losing efficacy in the subject.

In some embodiments of any of these methods, the subject has or is at risk for transthyretin-mediated amyloidosis (ATTR). In some embodiments of any of these methods, the subject has or is at risk for ATTR-cardiomyopathy or ATTR-polyneuropathy. In some embodiments of any of these methods, the subject has or is at risk for AL (light chain amyloidosis), Protein-energy malnutrition (PEM) or protein malnutrition, stroke, coronary disease, heart failure, Alzheimer's disease, vitreous floaters, an infectious disease such as tuberculosis, glaucoma, ocular myasthenia gravis, cerebral amyloid angiopathy, beta amyloid-related angiitis, atrial fibrillation, or leptomeningeal amyloidosis and variants like oculoleptomeningeal amyloidosis.

In some embodiments of any of these methods, the biological sample is a tissue sample. In some embodiments of any of these methods, the biological sample is a bodily fluid. In some embodiments of any of these methods, the bodily fluid is blood or a blood product such as serum or plasma, urine, cerebrospinal fluid, vitreous fluid, or a combination thereof.

In some embodiments of any of these methods, the bodily fluid is blood or a blood product such as serum or plasma, and wherein the critical threshold level is 18-20 mg/dL.

In some embodiments of any of these methods, the patient has been identified as having a low level of HMW TTR species.

In some embodiments of any of these methods, step (b) comprises using an antibody or other affinity ligand that specifically binds to TTR tetramer or HMW TTR varian or a methodology that preferentially isolates HMW TTR species. In some embodiments of any of these methods, step (b) comprises using turbidity-based or nephelometry-based methods.

In some embodiments of any of these methods, the therapeutic agent or therapeutic regimen comprises TTR stabilizing agents including: NSAIDs, AG10, tafamidis, diflunisal, anti-serum amyloid protein (SAP) -based approaches, CPHPC (miridesap)-based approaches, PRX004, doxycycline-based therapeutic agents, tetrabromobisphenol A (TBBPA), Tolcapone, Bacopa monnieri extract (BME), coumarin-based therapeutic agents, turmeric-based therapeutic agents, saponin-based therapeutic agents, flavonoid-based therapeutic agents, oleuropein aglycone (main phenolic component of extra virgin olive oil), other polyphenol-based therapeutic agents, green-tea (EGCG) based therapeutics, and other natural products with TTR binding/stabilizing properties, or combinations thereof. In some embodiments, the therapeutic agent or therapeutic regimen comprises gene-therapy approaches for increasing HMW TTR levels, or combinations thereof In some embodiments of any of these methods, the therapeutic agent or therapeutic regimen comprises TTR suppressing agents like patisiran, inotersen, or other TTR “knockdowns”, or combinations thereof. In some embodiments, the therapeutic agent is an agent that prevents destabilization of the TTR tetramer structure. In some embodiments, the therapeutic agent includes exogenous corticosteroids or anabolic steroids.

The major site of serum TTR synthesis is liver with normal concentration in the range of 0.2-0.4 mg/ml and half-life of 2 days. In the central nervous system, TTR is expressed in choroid plexus and is released into the cerebrospinal fluid with concentration in the range of 0.02-0.04 mg/ml (“Demonstration of transthyretin mRNA in the brain and other extrahepatic tissues in the rat,” J Biol Chem, 260, 11793-11798, 1985). In addition to plasma and cerebrospinal fluid, it is also expressed in the endothelial cells of Islets of Langerhans, retinal and ciliary pigment epithelia in trace amounts (“The retinal pigment epithelium is the unique site of transthyretin synthesis in the rat eye,” Invest. Ophthalmol. Vis. Sci. 31, 497-501, 1990; “Transthyretin synthesis in rabbit ciliary pigment epithelium,” Experimental Eye Research 81(2005) 306-312; “Widespread amyloid deposition in transplanted human pancreatic islets,” N Engl J Med. 2008 Aug. 28; 359(9):977-9. doi: 10.1056/NEJMc0802893). TTR may also undergo oligomerization and such TTR oligomers are specifically picked up by cardiomyocytes, neuronal and kidney cells leading to organ malfunctions (“Partial denaturation of transthyretin is sufficient for amyloid fibril formation in vitro,” Biochemistry. 1992 Sep. 15; 31(36):8654-60). Deficiency of the normal function of TTR has been known to be associated with obesity and diabetes (“Expression of an uncleavable n-terminal RASGAP fragment in insulin secreting cells increases their resistance towards apoptotic stimuli without affecting their glucose-induced insulin secretion,” JBC Papers in Press. Published on Jul. 25, 2005 as Manuscript M504058200). The roles of TTR in the central nervous system, especially in cognition and memory, psychological health and emotion have also been widely understood (“Transthyretin enhances nerve regeneration,” https://doi.org/10.1111/j.1471-4159.2007.04828.x; “Transthyretin: a key gene involved in the maintenance of memory capacities during aging,” Neurobiol Aging. 2008 November; 29(11):1721-32. Epub 2007 May 23). The oligomeric form of the TTR has been found to be involved in the pathophysiology of various diseases including atherosclerosis, familial amyloidosis polyneuropathy (“Amyloid fibril protein related to prealbumin in familial amyloidotic polyneuropathy,” Proc. Nat. Acad. Sci. U.S.A. 75:4499-4503, 1978), senile systemic amyloidosis (“Fibril in senile systemic amyloidosis is derived from normal transthyretin,” PNAS Apr. 1, 1990 87 (7) 2843-2845), familial amyloidosis cardiomyopathy (“Variant-sequence transthyretin (isoleucine 122) in late-onset cardiac amyloidosis in black Americans,” N Engl J Med. 1997; 336:466-73; “Discovery of γ-Mangostin as an Amyloidogenesis Inhibitor,” Scientific Reports volume 5, Article number: 13570 (2015); “Cavity filling mutations at the thyroxine-binding site dramatically increase transthyretin stability and prevent its aggregation,” Scientific Reports volume 7, Article number: 44709 (2017)) etc. Although the main function of TTR tetramer currently known is the transport of thyroxine and retinol bound to retinol binding protein (RBP), there are many other biological roles of TTR that are directly or indirectly related to antioxidant and oxidant properties and could be an important oxidative stress biomarker or therapeutic target. For instance, (i) TTR level correlates well with reactive oxygen species (ROS) or reactive nitrogen species (RNS) (“Effect of Nitric Oxide in Amyloid Fibril Formation on Transthyretin-Related Amyloidosis,” Biochemistry 2005, 44, 33, 11122-11129; “Transthyretin Aggregates Induce Production of Reactive Nitrogen Species,” Neurodegener Dis 2013;11:42-48); (ii) TTR gene expression is regulated by stress hormone, glucocorticoid and sex hormone, estradiol (“Neuronal production of transthyretin in human and murine Alzheimer's disease: is it protective?” J. Neurosci. 31 12483-12490, 2011; “Stress and Glucocorticoids Increase Transthyretin Expression in Rat Choroid Plexus via Mineralocorticoid and Glucocorticoid Receptors,” J Mol Neurosci (2012) 48:1-13); (iii) Even though TTR is an extracellular protein, it can induce oxidative stress in endoplasmic stress (ER) and hence involved in unfolded protein response (UPR) (“Endoplasmic Reticulum Stress Associated with Extracellular Aggregates,” The Journal of Biological Chemistry 281, 21998-22003, 2006; “Unfolded protein response-induced ERdj3 secretion links ER stress to extracellular proteostasis,” EMBO J. 2015 Jan. 2; 34(1): 4-19; “Endoplasmic reticulum proteostasis influences the oligomeric state of an amyloidogenic protein secreted from mammalian cells,” Cell Chem. Biol. 23, 1282-1293, 2016); (iv) The oligomeric forms of TTR also plays an important role in inducing oxidative stress and could be involved in different pathophysiologies (“Sequence-dependent denaturation energetics: A major determinant in amyloid disease diversity,” PNAS Dec. 10, 2002 99 (suppl 4) 16427-16432; “Age-related oxidative modifications of transthyretin modulate its amyloidogenicity,” Biochemistry. 2013 Mar. 19; 52(11):1913-26). In the light of these observations, this review article has been designed to discuss that TTR is associated with oxidative stress and could potentially be an effective biomarker of oxidative stress.

Throughout the present disclosure, the following abbreviations are used: Abbreviations: PA=prealbumin=PAB=transthyretin=TTR; HMW=higher molecular weight; AL=light-chain amyloidosis; ATTR-CM=amyloid transthyretin-cardiomyopathy; wtATTR-CM=wild-type ATTR-CM; AF=atrial fibrillation; LC=immunoglobulin light-chain; T4=thyroxine; CSF=cerebrospinal fluid.

The term “native” with respect to the structure transthyretin (TTR) refers to the normal folded structure of TTR in its properly functioning state (i.e., a TTR tetramer). As TTR is a tetramer in its natively folded form, non-native forms of TTR include, for example, misfolded TTR tetramers, TTR monomers, aggregated forms of TTR, and fibril forms of TTR. Non-native forms of TTR can include molecules comprising wild-type TTR amino acid sequences or mutations.

The term “misfolded” with respect to TTR refers to the secondary and tertiary structure of a TTR polypeptide monomer or multimer, and indicates that the polypeptide has adopted a conformation that is not normal for that protein in its properly functioning state. Although TTR misfolding can be caused by mutations in the protein (e.g., deletion, substitution, or addition), wild-type TTR proteins can also be misfolded in diseases, exposing specific epitopes. The term “pharmaceutically acceptable” means that the carrier, diluent, excipient, or auxiliary is compatible with the other ingredients of the formulation and not substantially deleterious to the recipient thereof.

Methods applicable to the present invention. Include mass spectroscopy, immuno-fixation, high-resolution melting analysis, fat pad biopsy—congo red, DPD-99mtc-scintigraphy, immunoelectron microsopy, conventional immunohistochemistry, laser microdiscetion+mass spectroscopy and immunoelectron microscopy, single radial immunodiffusion (SRID), ELISA, surface plasma resonance (SPR), and fluorescent probe exclusion assay (FPE).

In at least some embodiments, the invention preferably includes obtaining as fresh a biological sample as possible to minimize cell death and or non-specific protein degradation that may come about if a biological sample is not used immediately after being obtained, or after multiple freeze-thaws, or if subjected to hot temperatures, etc.

At least some embodiments follow NCCN guidelines, found at https://www2.tri-kobe.org/nccn/guideline/hematologic/english/amyloidosis.pdf, which define a hematologic complete response as normal kappa/lambda free light chain ratio).

### BRIEF DESCRIPTION OF THE DRAWINGS

Embodiments of the invention will be set forth in detail with reference to the drawings, in which:

Table 1 depicts the variation in serum TTR levels detected in several patients as measured using single radial immunodiffusion (SRID) or ELISA, as reported by Ando, Yukio, et al Biochemical and Biophysical Research Communications 1999.

FIGS. 1A and 1B are a series of graphs depicting (A) observed survival by enrollment level of prealbumin in 130 hemodialysis patients, and (B) observed survival by enrollment level of prealbumin in 128 peritoneal dialysis patients, as reported by Mittman et al., American Journal of Kidney Diseases, Volume 38, Issue 6, 1358-1364 2001.

FIGS. 2A and 2B are a series of graphs depicting Kaplan-Meier analysis for the event-free survival in (A) high TTR versus low TTR patients and (B) Group 0 versus Group L patients. Group L contains patients with low TTR levels and a low MNA score, as reported in Suzuki N, et al., Int Heart J (2014) 56: 226-33.

FIG. 3A is a graph depicting box and whisker plots demonstrating serum TTR levels stratified by the presence and type of atrial fibrillation (AF) in wild-type TTR amyloidosis. Serum TTR concentrations were lower in permanent or long-standing AF vs. paroxysmal or no AF, as reported in Mints et al., ESC Heart Failure 2018 5(5): 772-779.

FIG. 3B is a graph depicting survival in patients with atrial fibrillation (AF), stratified by TTR concentration that is above or below 180 μg/dL in ATTR wild-type amyloidosis, as reported in Mints et al., ESC Heart Failure 2018 5(5): 772-779.

FIG. 4 is a graph depicting Kaplan-Meier proportion of surviving patients receiving maintenance hemodialysis after 5 years of observation according to the 4 a prior-selected groups of serum prealbumin in 798 patients receiving maintenance hemodialysis, with the data adjusted for age and sex of patients, as reported in Rambod, M. et al, Am J Clin Nutr 2008 88(6): 1485-94.

FIG. 5 is a graph depicting the molar ratio (MR) for tafamidis (μM): TTR (μM) as a function of the percent stabilization of TTR. Percent stabilization represents TTR levels gained relative to levels after pharmacologic induced degradation. As demonstrated in Bulawa et.al 2016, when 14% TTR levels remain after pharmacologic degradation (aka new baseline), finding of ˜200% change in % TTR stabilization translates to ˜42% protein levels from new baseline levels. Similarly, 400% translates to ˜70% protein levels from new baseline, etc. See “Mechanism of Action and Clinical Application of Tafamidis in Hereditary Transthyretin Amyloidosis,” Neurol Ther. 2016 June; 5(1):1-25.

FIG. 6 is a graph depicting Kaplan-Meier analysis among 120 patients with TTR cardiac amyloidosis over the 1.9-year median follow-up for the outcome of death or orthotopic heart transplant (OHT), stratified by use of stabilizer.

FIG. 7 is a graph depicting Kaplan-Meier analysis among 120 patients with TTR-CA over the 1.9 (IQR 1.2-3.0) year median follow-up for the outcome of death or orthotopic heart transplantation (OHT), stratified by type of stabilizer.

Table 2 summarizes the current frequency of severe ocular abnormalities in ATTRV30M amyloidosis in amyloid centers worldwide, as reported by Buxbaum et al., Amyloid: The International Journal of Experimental and Clinical Investigation 2019 Jan 24.

FIG. 8 is a graph depicting survival stratified by BMI (in kg/m2) categories of <22 (solid line, n=25) or 22 (dashed line, n=81) or greater with the use of Kaplan Meier survival analysis. Each drop in probability curve indicates 1 event in that group. Vertical lines indicate censored patients, including those who were no longer followed but remained alive.

Table 3 is a table of AL clinical trials testing different chemotherapy and immunotherapies is shown below, including some that have already been FDA approved for related indications like multiple myeloma (MM).

Table 4 is a table showing the current frequency of several ocular abnormalities in ATTRV30M amyloidosis in amyloid centers worldwide in those untreated, post liver transplant, or post treatment with TTR stabilizer small-molecule tafamidis.

FIG. 9 is an exhibit depicting the prognostic value of the low pretreatment serum transthyretin level on overall survival (OS) in digestive cancers.

FIG. 10 is a graph depicting Kaplan-Meyer curves of post-resection disease-free survival in hepatocellular carcinoma patients with a normal (red colored, greater than or equal to 200 mg/L) or reduced preoperative serum prealbumin (blue colored; <200 mg/L), based on a cutoff value of 200 mg/L.

FIG. 11 is a graph depicting TTR levels for control, amnesic mild cognitive impairment (aMCI), and Alzheimer's disease (AD).

FIG. 12 (a) shows that plasma TTR from Alzheimer disease (AD) patients (n=10) are less stable than TTR from age-matched controls (n=10). (b) shows patients with AD have lower TTR folded/monomer TTR ratios compared to controls.

FIG. 13 is a plot showing that native TTR concentrations in the CSF are not significantly different between the tafamidis treated group, no tafamidis group, and non-ATTR controls if TTR concentrations are quantified using a fluorescence detection method employing a standard curve after TTRprotein is separated from other CSF components by ion-exchange chromatography using a covalent conjugate fluorogenic small molecule probe with affinity to TTR. Source: “Cerebrospinal Fluid and Vitreous Body Exposure to Orally Administered Tafamidis in hereditary ATTRV30M (p.TTRV50M) Amyloidosis Patients,” Amyloid. 2018 June; 25(2): 120-128.

FIG. 14 is a graph depicting a kinetic stabilizer effect on TTR located in the CSF after treatment with tafamidis by way of subunit exchange.

FIG. 15 is a graph showing that a study of 183 patients with idiopathic pulmonary fibrosis (IPF) revealed that those with baseline levels of at least 20 mg/dL or more had significantly greater survival-rates compared to those with levels below 20 mg/dL (p<0.001).

FIG. 16 is a graph showing that a 7 retrospective study of 102 heatstroke patients admitted to the ICU showed that patients with TTR (PA=prealbumin) levels less than or equal to 15 mg/dL had a lower survival rate compared to patients with levels greater than 15 mg/dL (p<0.001).

Table 5 is a table showing analysis of basic variables and parameters of three patient groups.

FIG. 17 is a graph showing that in a study that enrolled 81 ischemic stroke patients, serum TTR levels at discharge were significantly lower in non-survivors compared to survivors (p=0.009).

Table 6 is a table showing clinical data relating to Guillain-Barré syndrome, chronic inflammatory demyelinating polyneuropathy, multiple sclerosis, and other neurological diseases. Highlighted rows indicate TTR levels in the CSF or plasma.

FIG. 18 is a graph showing that a retrospective analysis of 146 patients with biopsy-proven wtATTR-CM performed results revealed significantly lower serum TTR levels were found in those with atrial fibrillation (AF) vs those without AF (p<0.005).

FIG. 19 is a graph showing that amongst 95 ATTRwt patients, those with arrhythmia were found to have significantly lower serum TTR levels compared to those without arrhythmia (p=0.0058).

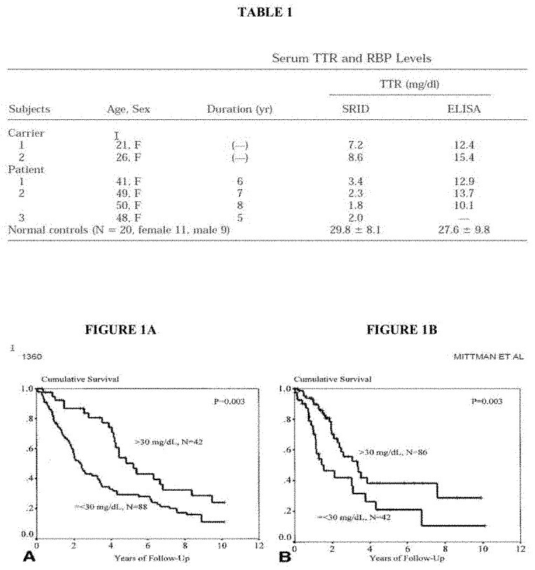

### DETAILED DESCRIPTION OF THE PREFERRED EMBODIMENTS

Embodiments of the invention will now be set forth with reference to the drawings.

The invention in some embodiments provides methods for measuring higher molecular weight transthyretin (TTR) species that correlate to clinically relevant characteristics such as disease severity, ideal therapeutic regimen, prognosis, and assessment of the effectiveness of a therapeutic regimen. Higher molecular weight (HMW) TTR species includes TTR tetramers or a mixture of TTR species where the predominant species in a biological sample are TTR tetramers. TTR levels obtained by these methods enable these levels to be used as a biomarker, which can be used for multiple purposes as described below.

While the examples provided herein at times reference “TTR tetramers” or “functional equivalents” of TTR levels, the skilled artisan will appreciate that the following methods can also be used to detect any higher molecular weight TTR species, including, by way of non-limiting example, non-TTR tetramers that are also higher molecular weight, TTR tetramers, combinations thereof, and/or other any other combination where TTR tetramers are a predominant TTR species, including wild-type,mutant, truncates, post-translationally modified, or other variant TTR species.

High molecular weight TTR species, such as for example, TTR tetramers or combinations in which TTR tetramers are the predominant species, are preferably measured from a biological taken from a subject. In some embodiments, the biological sample is a bodily fluid from a subject. As used herein, a “bodily fluid” refers to a sample of fluid isolated from anywhere in the body of the subject, including but not limited to, for example, blood, plasma, serum, urine, sputum, spinal fluid, pleural fluid, nipple aspirates, lymph fluid, fluid of the respiratory, intestinal, and genitourinary tracts, ocular related fluids (aqueous humor, vitreous fluid, tear fluid), saliva, breast milk, fluid from the lymphatic system, semen, cerebrospinal fluid, intra-organ system fluid, ascitic fluid, tumor cyst fluid, amniotic fluid, and combinations thereof

As used herein, the term “functional equivalents” of TTR levels, includes anything related to exploiting higher molecular weight TTR species functions (i.e., protease, binding partner interaction with Apo-L). It is also to be understood that while the exact mechanism for how more TTR tetramers and/or higher MW TTR species translate into favorable clinical benefit is a work in progress, strong preclinical work has already demonstrated higher TTR MW species (i.e., TTR tetramer) can act functionally as a scavenger of misfolded TTR monomers. Other work has demonstrated TTR tetramer is able to perform cleavage of misfolded beta amyloid units and/or misfolded lower MW TTR species. In essence, TTR tetramers can protect against numerous amyloid diseases where the amyloid source can come from any different precursor proteins including TTR. To this end, our discovery has direct and broad relevance across a range of areas in medicine including neurology, ophthalmology, cardiology, orthopedics, and more. For neurology, this includes diseases like Alzheimer's disease and/or other diseases where amyloid or prion deposition have been implicated. In fact, compared to healthy controls, Alzheimer patients have been shown to have reduced TTR tetramer levels in the CSF (Riisoen, H. 1988 Reduced prealbumin in CSF of severely demented patients with AD. Acta Neuro. Scan. 78).

The methodologies provided herein are useful for identifying and measuring a higher molecular weight transthyretin (TTR) species, such as, for example, TTR tetramer levels, which can then provide diagnostic and/or be prognostic of clinically relevant functional outcomes for a number of diseases, including those that cause significant morbidity and mortality. The invention provides methodologies that enable physiological TTR tetramer levels to be directly clinically informative. In contrast, levels from other measurement tools, which are less biased or otherwise sensitive for measuring tetramer levels and/or include measurement of TTR lower molecular weight species, should be interpreted as not necessarily falling under the proposed algorithms/discussion provided herein. The working examples provided herein also provide disease specific clinical datasets that support the use of TTR tetramer levels in the clinical realm including by way of disease detection, risk-stratification, treatment responsiveness, disease progression, and/or prophylaxis. By using available clinical datasets to identify TTR tetramer level thresholds, these algorithms provide clinicians tools for how to use this important biomarker properly so as when obtained/used properly, measured levels are directly correlated with clinical 8outcomes, in both directions (aka lower levels correlate with poorer outcomes and higher levels correlate with improved outcomes).

Utilization of specific assays for measuring TTR tetramer levels allow for clinically useful information to be obtained in subjects at risk of or suffering from diseases that may or may not be currently linked to transthyretin (TTR). The methods provided herein are useful to identify patients having low TTR tetramer levels, regardless of whether they are suffering from a TTR-related or associated disorder. The methods provided herein are also useful in asymptomatic patients who report to be healthy, but have lower TTR tetramer levels that may put them at increased risk for a poor outcome from any number of diseases. For example, the methods provided herein are useful in identifying which elderly populations, healthy or not, that may be at increased risk for morbidity and mortality.

The ability to reliably detect clinically useful TTR tetramer levels in patients provides critical, real-time information for medical professionals. Identification of patients who have TTR tetramer levels below a critical threshold level may be at an increased risk of heart failure or death. Studies with patients having various diseases such as renal disease, cardiomyopathy, or malnutrition have shown that this threshold level in the plasma is in the lower normal range of about 18-20 mg/dL. For example, in embodiments where the biological sample from the patient is blood or a blood product such as serum or plasma, the critical threshold level is 18-20 mg/dL. This critical threshold level can be calculated for other sources of biological samples. If other critical threshold levels are determined for use in other embodiments, such other critical threshold levels should be considered to be within the scope of the invention.

A patient with TTR tetramer levels above 18-20 mg/dL in blood, plasma, or serum is generally considered to have HMW TTR levels at the lower end of normal levels and is less likely to be at risk for heart failure or death compared to someone with lower levels. A patient with TTR tetramer levels below 18-20 mg/dL in blood, plasma, or serum is considered to have low HMW TTR levels and is at risk of a poor clinical outcome, including heart failure or death. When a patient has been identified as having low HMW TTR levels, each 1 mg/dL drop in detected TTR tetramer level in a blood or blood product samples from that patient increases the risk of death by about 7 to 11%.

In some embodiments, the methods provided herein include the steps of (a) providing a biological sample from the subject, and (b) detecting a level of transthyretin (TTR) tetramer present in the biological sample, wherein a detected level of HMW TTR species in the biological sample below a critical threshold indicates an increased risk of a poor clinical outcome, including heart failure or death. For example, in embodiments where the biological sample is blood or a blood product such as serum or plasma, the critical threshold level is 18-20 mg/dL.

In some embodiments, the methods provided herein include the steps of (a) providing a biological sample from the subject, (b) detecting a level of transthyretin (TTR) tetramer present in the biological sample, and (c) comparing the detected level of TTR tetramer in the biological sample to a control sample of TTR tetramer in a control sample from a control subject who is not suffering from or at risk for TTR amyloid cardiomyopathy, wherein a decrease in the detected level of HMW TTR in the biological sample as compared to the control level of HMW TTR in the control sample indicates an increased risk of a poor clinical outcome.

In some embodiments, step (b) comprises using an antibody or other affinity ligand that specifically binds to TTR tetramer or another HMW TTR variant, a methodology that preferentially isolates HMW TTR species, such as, for example, liquid chromatography. It may be possible for methods that measure tetramer specific functions to be used, including whether estimating changes in tetramer levels and/or absolute levels.

In some embodiments, step (b) comprises using turbidity-based or nephelometry-based methods. There are several current methods of detecting TTR that should not be used in the methods provided herein, such as for example, subunit exchange or other kinetic exchange assays, western blot assays, ELISA, tetramerization, or stabilization assays, fluorescence probe exclusion (FPE) assays, radial immunodiffusion assays (RIA), or any combination thereof. Current forms and/or use of these assays and their associated antibodies or other binding agents (if applicable), do not appear to provide direct measurements of absolute TTR tetramer levels in the biological sample.

Regardless of the disease or other indication, the methods provided herein demonstrate that a detected level of HMW TTR in a biological sample of less than a certain threshold indicates increased risk for poorer clinical outcomes generally. For serum derived biological samples, a range of about 18-20 mg/dL indicates a starting reference point where subjects may start to be at increased risk of a clinical outcome related to a diagnosed or an undiagnosed disease. TTR tetramer levels at or below 5 mg/dL are considered grave risks of death, and the patient should be seen by a medical specialist for immediate intervention such as ICU care, parental feeding, and so on.

Responsiveness to treatment initiation may be gauged by serial measurements of TTR tetramer such that increases in HMW TTR levels can be viewed as treatment responders whereas those with decreasing TTR tetramer levels may be viewed as non-responders.

In some embodiments, the biological sample is a bodily fluid, such as, for example, blood or a blood product such as serum or plasma, urine, cerebrospinal fluid, vitreous fluid, or a combination thereof In some embodiments, the biological sample is a tissue sample.

In some embodiments, step (a) further comprises the step of isolating the TTR tetramer from other TTR isoforms in the biological sample.

The invention also provides methods of diagnosing risk of heart failure or death in a subject at risk for or suffering from a TTR-related or TTR-associated disease or disorder, the method comprising: (a) providing a biological sample from the subject, and (b) detecting a level of transthyretin (TTR) tetramer present in the biological sample, wherein a detected level of TTR tetramer in the biological sample below a critical threshold indicates an increased risk of heart failure or death in the subject. For example, in embodiments where the biological sample is blood or a blood product such as serum or plasma, the critical threshold level is 18-20 mg/dL

The invention also provides methods of diagnosing risk of heart failure or death in a subject at risk for or suffering from a TTR-related or TTR-associated disease or disorder, the method comprising: (a) providing a biological sample from the subject, (b) detecting a level of transthyretin (TTR) tetramer present in the biological sample, and (c) comparing the detected level of TTR tetramer in the biological sample to a control level of TTR tetramer in a control sample from a control subject who is not suffering from or at risk for the TTR-related or TTR-associated disease or disorder, wherein a decrease in the detected level of TTR in the biological sample as compared to the control level of TTR tetramer in the control sample indicates an increased risk of heart failure or death in the subject.

In some embodiments, the TTR-related or associated disease or disorder is transthyretin-mediated amyloidosis (ATTR), which is generally associated with cardiomyopathy and/or polyneuropathy. In some embodiments, the TTR-related or associated disease or disorder is ATTR-cardiomyopathy, ATTR-polyneuropathy, AL (light chain amyloidosis), protein malnutrition, stroke, coronary disease, heart failure, Alzheimer's disease, vitreous floaters, an infectious disease such as tuberculosis, glaucoma, ocular myasthenia gravis, cerebral amyloid angiopathy, beta amyloid-related angiitis, atrial fibrillation, or leptomeningeal amyloidosis and variants including oculoleptomeningeal amyloidosis.

In some embodiments, the patient is a healthy elderly patient at increased risk of death if lower TTR tetramer levels are detected. In some embodiments, the method is used as a prognostic for the success rate of liver transplants. In some embodiments, the method is used on a patient on hemodialysis or peritoneal dialysis (any disease leading to end stage renal failure or equivalent thereof).

In some embodiments, step (b) comprises using turbidity-based or nephelometry-based methods. There are several current methods of detecting TTR that should not be used in the methods provided herein, such as for example, subunit exchange or other kinetic exchange assays, western blot assays, ELISA, tetramerization, or stabilization assays, fluorescence probe exclusion (FPE) assays, radial immunodiffusion assays (RIA), or any combination thereof. Current forms and/or use of these assays and their associated antibodies or other binding agents (if applicable), do not appear to provide direct measurements of absolute TTR tetramer levels in the biological sample.

Regardless of the disease or other indication, the methods provided herein demonstrate that a detected level of TTR tetramer in a biological sample of less than a certain threshold indicates increased risk for poorer clinical outcomes generally. For serum derived biological samples, a range of about 18-20 mg/dL indicates a starting reference point where subjects may start to be at increased risk of a clinical outcome related to a diagnosed or an undiagnosed disease. TTR tetramer levels at or below 5 mg/dL are considered grave risks of death, and the patient should be considered to be seen by a medical specialist for immediate intervention such as ICU care, parental feeding, and so on.

In some embodiments, the biological sample is a bodily fluid, such as, for example, blood or a blood product such as serum or plasma, urine, cerebrospinal fluid, vitreous fluid, or a combination thereof In some embodiments, the biological sample is a tissue sample.

In some embodiments, step (a) further comprises the step of isolating the TTR tetramer from other TTR isoforms in the biological sample.

The methods provided herein are also useful for predicting risk of heart failure or death in a subject at risk for or suffering from a TTR-related or TTR-associated disease or disorder, the method comprising: (a) providing a biological sample from the subject, and (b) detecting a level of transthyretin (TTR) tetramer or HMW TTR levels present in the biological sample, wherein the detected level of TTR in the biological sample is used to stratify the patient into a category of a poor clinical outcome, including risk of heart failure or death based on the detected level of HMW TTR species.

The methods provided herein are also useful for predicting risk of heart failure or death in a subject at risk for or suffering from a TTR-related or TTR-associated disease or disorder, the method comprising: (a) providing a biological sample from the subject, (b) detecting a level of HMW TTR levels present in the biological sample, and (c) comparing the detected level of TTR tetramer in the biological sample to a control level of TTR tetramer in a control sample from a control subject who is not suffering from or at risk for TTR amyloid cardiomyopathy, wherein a decrease in the detected level of TTR in the biological sample as compared to the control level of TTR tetramer is used to stratify the patient into a category of risk of heart failure or death based on the detected level of TTR tetramer.

For example, in some embodiments, the TTR-related or TTR-associated disease or disorder is ATTR-cardiomyopathy (wild-type), and each 1 mg/dL increase in detected TTR tetramer level in a blood or blood product sample reduces the risk of death by about 7 to 11% in the patient.

In some embodiments, the TTR-related or associated disease or disorder is transthyretin-mediated amyloidosis (ATTR), which is generally associated with cardiomyopathy and/or polyneuropathy. In some embodiments, the TTR-related or associated disease or disorder is ATTR-cardiomyopathy, ATTR-polyneuropathy, AL (light chain amyloidosis), Protein energy malnutrition (PEM), or protein malnutrition, stroke, coronary disease, heart failure, Alzheimer's disease, vitreous floaters, an infectious disease such as tuberculosis, glaucoma, ocular myasthenia gravis, cerebral amyloid angiopathy, beta-amyloid related angiitis, atrial fibrillation, or leptomeningeal amyloidosis and variants including ocular leptomeningeal amyloidosis.

In some embodiments, the patient is a healthy elderly patient at increased risk of death if lower TTR tetramer levels are detected. In some embodiments, the method is used as a prognostic for the success rate of liver transplants. In some embodiments, the method is used on a patient on hemodialysis or peritoneal dialysis (aka any disease leading to end stage renal failure or equivalent thereof).

In some embodiments, step (b) comprises using an antibody or other affinity ligand that specifically binds to TTR tetramer or a methodology that preferentially isolates higher TTR molecular weight species, such as, for example, liquid chromatography. It may be possible for methods that measure tetramer specific functions to be used, including whether estimating changes in tetramer levels and/or absolute levels.

In some embodiments, step (b) comprises using turbidity-based or nephelometry-based methods. There are several current methods of detecting TTR that should not be used in the methods provided herein, such as for example, subunit exchange or other kinetic exchange assays, western blot assays, ELISA, tetramerization stabilization assays, fluorescence probe exclusion (FPE) assays, radial immunodiffusion assays (RIA), or any combination thereof While some ELISA kits may correlate with turbidity/nephelometry based assays, many do not and may have resulted in many conflicting reports. Current forms and/or use of these assays and their associated antibodies or other binding agents (if applicable), do not appear to provide direct measurements of absolute TTR tetramer levels in the biological sample.

Regardless of the disease or other indication, the methods provided herein demonstrate that a detected level of TTR tetramer in a biological sample of less than a certain threshold indicates increased risk for poorer clinical outcomes generally. For serum derived biological samples, a range of about 18-20 mg/dL indicates a starting reference point where subjects may start to be at increased risk of a clinical outcome related to a diagnosed or an undiagnosed disease. TTR tetramer levels at or below 5 mg/dL are considered grave risks of death, and the patient should be seen by a medical specialist for immediate intervention such as ICU care, parental feeding, and so on.

In some embodiments, the biological sample is a bodily fluid, such as, for example, blood or a blood product such as serum or plasma, urine, cerebrospinal fluid, vitreous fluid, or a combination thereof In some embodiments, the biological sample is a tissue sample.

In some embodiments, step (a) further comprises the step of isolating the TTR tetramer from other TTR isoforms in the biological sample.

The invention also provides methods of treating or delaying the progression of transthyretin (TTR) amyloid cardiomyopathy or preventing heart failure in a subject at risk for or suffering from TTR amyloid cardiomyopathy, the method comprising: (a) providing a biological sample from the subject, (b) detecting a level of transthyretin (TTR) tetramer present in the biological sample, (c) detecting whether there is a decreased level of TTR tetramerization in the subject by comparing the detected level of TTR tetramer in the biological sample to a control level of TTR tetramer in a control sample from a control subject who is not suffering from or at risk for TTR amyloid cardiomyopathy, and (d) administering a therapeutically effective amount of a therapeutic agent to increase level of the TTR tetramerization in the subject when there is a decrease in the detected level of TTR in the biological sample.

In some embodiments of any of these methods, the therapeutic agent or therapeutic regimen comprises TTR stabilizing agents including: NSAIDs, AG10, tafamidis, diflunisal, anti-serum amyloid protein (SAP)-based approaches, CPHPC (miridesap)-based approaches, PRX004, doxycycline-based therapeutic agents, tetrabromobisphenol A (TBBPA), Tolcapone, Bacopa monnieri extract (BME), coumarin-based therapeutic agents, turmeric-based therapeutic agents, saponin-based therapeutic agents, flavonoid-based therapeutic agents, oleuropein aglycone (main phenolic component of extra virgin olive oil), other polyphenol-based therapeutic agents, green-tea (EGCG) based therapeutics, and other natural products with TTR binding/stabilizing properties, or combinations thereof. In some embodiments, the therapeutic agent or therapeutic regimen comprises gene-therapy approaches for increasing HMW TTR levels, or combinations thereof. In some embodiments of any of these methods, the therapeutic agent or therapeutic regimen comprises TTR suppressing agents like patisiran, inotersen, or other TTR “knockdowns”, or combinations thereof. In some embodiments, the therapeutic agent is an agent that prevents destabilization of the TTR tetramer structure. In some embodiments, the therapeutic agent includes exogenous corticosteroids or anabolic steroids.

The invention also provides methods of treating or delaying the progression of familial amyloid polyneuropathy or preventing heart failure in a subject at risk for or suffering from familial amyloid polyneuropathy, the method comprising: (a) providing a biological sample from the subject, (b) detecting a level of transthyretin (TTR) tetramer present in the biological sample, (c) detecting whether there is an increased level of TTR tetramerization in the subject by comparing the detected level of TTR tetramer in the biological sample to a control level of TTR tetramer in a control sample from a control subject who is not suffering from or at risk for TTR amyloid cardiomyopathy, and (d) administering a therapeutically effective amount of a therapeutic agent to decrease the level of TTR tetramerization in the subject when there is an increase in the detected level of TTR in the biological sample.

The invention also provides methods of monitoring the efficacy of a therapeutic regimen in a person at risk for or suffering from transthyretin (TTR) amyloid cardiomyopathy, the method comprising: (a) providing a first biological sample from the subject at a first time point, (b) detecting a first level of transthyretin (TTR) tetramer present in the first biological sample, (c) providing a second biological sample from the subject at a second time point, (d) detecting a second level of TTR tetramer present in the second biological sample, and (e) comparing the first detected level of TTR tetramer to the second detected level of TTR tetramer, wherein a decrease in the second detected level of TTR as compared to the first detected level of TTR tetramer indicates that the therapeutic regimen is losing efficacy in the subject.

In some embodiments, the therapeutic regimen comprises administration of tafamidis. In some embodiments, the therapeutic regimen comprises administration of AG10.

The invention also provides methods of monitoring the efficacy of a therapeutic regimen in a person at risk for or suffering from familial amyloid polyneuropathy, the method comprising: (a) providing a first biological sample from the subject at a first time point, (b) detecting a first level of transthyretin (TTR) tetramer present in the first biological sample, (c) providing a second biological sample from the subject at a second time point, (d) detecting a second level of TTR tetramer present in the second biological sample, and (e) comparing the first detected level of TTR tetramer to the second detected level of TTR tetramer, wherein a decrease in the second detected level of TTR as compared to the first detected level of TTR tetramer indicates that the therapeutic regimen is losing efficacy in the subject.

In some embodiments, the therapeutic regimen comprises administration or co-administration of patisiran or another TTR knock-down agent (i. e, inotersen). Other TTR suppressors are in development and can be used in at least some embodiments of the invention.

The invention also provides methods of screening TTR tetramer as a biomarker for a disease or disorder, the method comprising: (a) providing biological samples from patients at risk or suffering from the disease or disorder; (b) detecting the level of TTR tetramer in each biological sample from each patient; and (c) correlating the detected level of TTR tetramer in each biological sample from each patient with disease status.

The invention also provides methods of selecting a treatment regimen by stratifying patient population based on TTR tetramer levels.

The term “subject” used herein is intended to include all mammals, including a human, non-human primate, companion animal (e.g., cat, dog, horse), farm animal, work animal, or zoo animal. In some embodiments, the subject is a human. In some embodiments, the subject is a companion animal. In some embodiments, the subject is an animal in the care of a veterinarian. Preferably, the subject is a human.

“Patient” refers to a human diagnosed with a disease or condition that can be treated in accordance with the inventions described herein. In some embodiments it is contemplated that the formulations described herein may also be used in other mammals. The terms “subject” and “patient” are used interchangeably herein.

The terms “treatment” and “treating” a patient refer to reducing, alleviating, stopping, blocking, or preventing the symptoms of a disorder, disease, or other affliction in a patient. As used herein, “treatment” and “treating” includes partial alleviation of symptoms as well as complete alleviation of the symptoms for a time period. The time period can be hours, days, months, or even years.

By an “effective” amount or a “therapeutically effective amount” of a drug or pharmacologically active agent is meant a nontoxic but sufficient amount of the drug or agent to provide the desired effect, e.g., treatment of depression. An appropriate “effective” amount in any individual case may be determined by one of ordinary skill in the art using routine experimentation.

Also, any and all alterations and further modifications of the invention, as would occur to one of ordinary skill in the art, are intended to be within the scope of the invention.

All references, patents, patent applications or other documents cited are hereby incorporated by reference in their entireties into the present disclosure.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Assay Dependency of TTR Levels from Same Patients

One study used two different methods to measure TTR levels in either asymptomatic or symptomatic hereditary ATTR patients with Y114C amino acid change (see Ando, Yukio, et al Biochemical and Biophysical Research Communications 1999). The two methods, single radial immunodiffusion (SRID) and enzyme-linked immunosorbent assay (ELISA), despite being run on samples from the same patients, measured significantly different TTR levels. In carriers (asymptomatic), SRID vs ELISA measured mean TTR levels of 7.9 mg/dL vs 13.9 mg/dL, respectively. In symptomatic patients, SRID vs ELISA measured mean TTR levels of 2.3 mg/dL (n=4) vs 12.2 mg/dL (n=3), respectively (Table 1). This is in contrast to normal controls, where both assays measured similar TTR values that were also higher (˜29.8 mg/dL). These findings are important because they not only demonstrate the impact which assay used can have on TTR levels, but they show how diseased populations relative to healthy-controls can have different TTR isoform ratios which impact TTR level measurements differently depending on the assay. In another study by Haagsma et al. ([1997]. Amyloid: Int. J. Exp. Clin. Invest. 4, 112-117) and by Ueno et al. ([1992] Brain 115, 1275-1289), similarly low TTR levels (closer to 7.9 vs 13.9 mg/dL) in patients with the same mutation were also measured using nephelometry. However, the authors concluded immunodiffusion based techniques (SRID) could not properly determine TTR serum levels in Y114C carriers. This is important because the discovery involved in the present invention states otherwise and that these levels are reflective of HMW TTR levels. Also, the similar low TTR levels measured across two different publications with two different assays supports our discovery findings in that consistency in TTR levels can be achieved when using correct methods.

A separate paper by Sekijima Y et. al (2015) studied a patient with a Y114H mutation, (same amino acid location on TTR as Y114C patient but with different mutation and amino acid). Findings included that this patient also presented with very low HMW TTR levels (9.3 mg/dL by immunoturbidity, SRL Inc) (Sekijima et al., Journal of the Peripheral Nervous System 20:372-379 (2015) Research Report). Conclusions from these authors included that this unstable variant TTR protein was likely degraded in the endoplasmic reticulum before reaching a patient's blood, which was why HMW TTR levels in this patient were measured as very low and similar to levels in the other study measured by SRID.

The totality of the aforementioned findings demonstrated that TTR levels measured by SRID or immunoturbidity, as reported by Ando, Yukio, et al (1999) or by Sekijima Y et. al (2015), respectively indeed measured HMW TTR levels whereas the comparatively higher TTR levels measured by ELISA (see Ando, Yukio, et al (1999) may have measured smaller TTR species (e.g., monomers) in addition to HMW TTR species. Our discovery is critical here because it identifies the ELISA method or antibody used therein, or both, as the source for the relatively higher measured TTR levels and not the TTR levels themselves being unreliable. In addition, using clinical data, our discovery identifies assays) that measure HMW TTR levels which can be used as a biomarker. Examples include turbidimetry, nephelometry, immunodiffusion-based methods.

### Example 2

### Cancer Relevance; Improved Survival for AL with Greater HMW TTR Levels

Immunoglobulin light chain amyloidosis (AL) is both a cancer and a proteinopathy. AL is associated with other cancers that also produce clonal immunoglobulin light chains (LC) of kappa or lambda type including multiple myeloma and monoclonal gammopathy of undetermined significance (MGUS). AL is also associated with B-cell lymphomas and Waldenstrom macroglobulinemia. Given similar pathophysiology between AL and MM, it is not surprising that treatments which work for multiple myeloma also work for AL. Of note, despite AL being a cancer, the most common cause of death in this population is cardiac-related. The most common causes of death in ATTR-cardiomyopathy (ATTR-CM) is also cardiac-related. A recent publication comparing ATTR-CM and AL showed that AL patients have a significantly greater risk of death compared to ATTR-CM (HR 3.02, 2.47-3.7, p<0.001) (see Xin et al., “Prognostic impact of light-chain and transthyretin-related categories in cardiac amyloidosis: a systematic review and meta-analysis.” Hellenic Journal of Cardiology 2019, Feb. 8).

Treatment options for AL include autologous stem-cell transplant (ASCT) for eligible patients and for transplant-ineligible patients' different chemotherapy regimens. Immunotherapy options for AL mainly include immunomodulatory agents and monoclonal antibodies, with immunomodulatory agents often included within different chemotherapy regimens. https://www.cancer.net/cancer-types/amyloidosis/treatment-options For amyloidosis, targeted therapies include anti-angiogenesis therapy, monoclonal antibodies, and proteasome inhibitors. Immunomodulatory drugs include lenalidomide (Revlimid), pomalidomide (Pomalyst), and thalidomide (Synovir, Thalomid). A monoclonal antibody is a type of targeted therapy. It recognizes and attaches to a specific protein in the abnormal cells, and it does not affect cells that don't have that protein. Drugs in this class include daratumumab (Darzalex) and elotuzumab (Empliciti). A second type of monoclonal antibody directly targets the amyloid itself There are currently several of these antibodies in clinical trials. For example, some of the chemotherapy regimens used in AL include: [1] cyclophosphamide, bortezomib, dexamethasone (CyBorD), [2] bortezomib, melphalan, dexamethasone, [BMDex], [3] bortezomib, prednisone, cyclophosphamide, and thalidomide [RPCD]. Immunomodulatory agents include thalidomide and analogues like lenalidomide or pomalidomide. The FDA recently approved daratumumab in combination with bortezomib, thalidomide, and dexamethasone for newly diagnosed multiple myeloma (MM) patients who are eligible for ASCT (Sep. 26, 2019). In addition to immune modulatory agents, treatment regimens with monoclonal antibodies like daratumumab (anti-CD38), isatuximab (anti-CD38) or elotuzumab (anti-SLAMF7) have already demonstrated impressive response-rates and improved progression free survival (PFS) rates in multiple myeloma (MM) clinical trials leading to approval. These agents have also demonstrated promising efficacy/safety in early AL clinical studies and are currently being investigated in different late-stage clinical trials for AL (i.e., Phase 3 ANDROMEDA trial evaluating Daratumumab plus CyBorD). Other immunotherapy strategies in development include: anti-human-serum amyloid P component (SAP) and anti-light chain monoclonal antibodies (i.e, CAEL-101, previously known as 11-1F4). While different immunotherapy and chemotherapy AL trials are ongoing, some may have failed due to the design of primary endpoints with poor biomarker sensitivity (i. e, NEOD001). While this is outside the scope of this patent, our discovery offers a new biomarker for patients with a lethal cancer type where currently used biomarkers for gauging treatment response (i. e, NT-proBNP, eGFR (estimated glomerular filtration rate)) are non-specific for cancer and amyloid. A table of AL clinical trials testing different chemotherapy and immunotherapies is shown below, including some that have already been FDA approved for related indications like MM. These are shown in Table 3, taken from “Emerging Therapeutics for the Treatment of Light Chain and Transthyretin Amyloidosis,” JACC Basic Transl Sci. 2019 Jun. 24; 4(3):438-448. doi: 10.1016/j.jacbts.2019.02.002. eCollection 2019 June.

Our discovery introduces a biomarker for AL patients with established clinical utility that could help with earlier diagnosis or gauge treatment responsiveness to treatment options currently available for AL. Though TTR levels have never been the primary focus in AL or associated conditions, therapeutics that stabilized HMW TTR species and provided clinical benefit for ATTR patients have also delivered a clinical benefit in AL patients too. We think such findings make our discovery timely and relevant for AL and related conditions especially since AL patients are a higher-risk population that have been found to have a significantly greater risk of death compared to ATTR-CM patients. Furthermore, AL patients are often initially misdiagnosed, with diagnosis more than 1 year after symptom onset in ˜40% of cases (affected patients die within a few months).

In a study of 106 patients with histologically confirmed AL, patients with serum TTR levels greater than or equal to 20 mg/dL had significantly longer survival compared to patients with less than 20 mg/dL (p=0.01) (see Caccialanza, Riccardo, Nutritional status of outpatients with systemic . . . Am. J. Clin Nutr 2006). Multivariate cox regression analysis demonstrated serum prealbumin was an independent predictor of survival (HR 0.86, 0.79-0.94; p=0.001), whereas serum albumin and transferrin were not predictors. Serum HMW TTR levels were measured by immunonephelometry, which is a method that our patent discovery states measures HMW TTR levels which are clinically useful as a biomarker (Dade Behring).

A case report was published that discussed a 45-year-old man who was transferred to the emergency department for sudden left upper quadrant pain, found to be in hemorrhagic shock, and diagnosed with AL with multi-organ involvement. Notably, after four courses of RPCD chemotherapy treatment, TTR levels increased by 34% to 17.05 mg/dL (from 11.23 mg/dL). HMW TTR levels were measured by immunonephelometry (Dade Behring, Marburg, Germany). The patient was noted as being alive one-year after treatment initiation (or two years after symptom onset), with near normalization of kappa light chain levels (462.5 mg/L to 34.8 mg/L) and no longer having elevated troponin-I levels. The median survival rate in untreated AL patients with cardiac involvement is ˜6 months following symptom onset. “Multiorgan involvement by amyloid light chain amyloidosis,” J Int Med Res. 2019 April; 47(4): 1778-1786.

Overall, this discovery provides a new biomarker that has immediate clinical utility (one-time or serial monitoring in patients on or off the standard of care) in AL and associated conditions. Similar findings of reduced HMW TTR levels compared to controls have been found in other amyloid conditions (not ATTR or AL), like amyloid A (AA) amyloidosis, which is the most common form of systemic amyloidosis with the main fibril protein AA and fragments of serum amyloid A (SAA) protein. “Serum prealbumin and retinol-binding protein in the prealbumin-related senile and familial forms of systemic amyloidosis,” Lab Invest. 1985 March; 52(3):314-8.

### Example 3

### ATTR; Evaluation of TTR Stabilizers in ATTR Clinical Trials

We believe our discovery has relevance to the recently reported Phase 3 ATTR-ACT trial that readout positive mortality reduction with pooled tafamidis (20 mg and 80 mg QD) vs placebo. Specifically, given TTR tetramer levels have not yet been released publicly, our proprietary analysis suggests minimal (˜1 mg/dL) differences in TTR levels were detected between the two different dose-strengths (20 vs 80 mg). This is in line with one of Pfizer's expectations for the Phase 3 protocol (Maurer 2018, Phase 3 Final Protocol, 31 Jul. 2013. PF-06291826 Tafamidis Meglumine. B3461028) when looking at the plot of % TTR stabilization (y-axis) and molar ratio of tafamidis in FIG. 5: TTR levels (μM). We highlighted in red the wide ranges for pharmacodynamic effects related to 80 mg relative to 20 mg (in blue shade) to point out (1) potential for both dose-strengths to nearly overlap, (2) plotted data points were from Phase 1 data in healthy volunteers using liquid formulations of tafamidis (not capsule formulation given in Phase 3 ATTR-ACT trial), and (3) 80 mg PD expectations were calculated as a 4× multiple of 20 mg PD data, which could be important given PK variations may have not been optimized and could explain differences seen in Phase 3 ATTR-ACT clinical trial which tested 20 mg and 80 mg doses of tafamidis.

### Example 3

### ATTR, Hemodialysis, Acute Heart Failure; Lower HMW TTR Levels Correlate with Poorer Clinical Outcomes

The methods provided herein demonstrate that a minimum TTR tetramer concentration and/or its functionally equivalent in a biological sample is needed such that less than a certain threshold indicates increased risk of a poor clinical outcome. Using such methods, for serum derived biological samples, levels of 18-20 mg/dL provide a starting threshold level where subjects may be at increased risk of a poor clinical outcome related to a diagnosed or an undiagnosed disease.

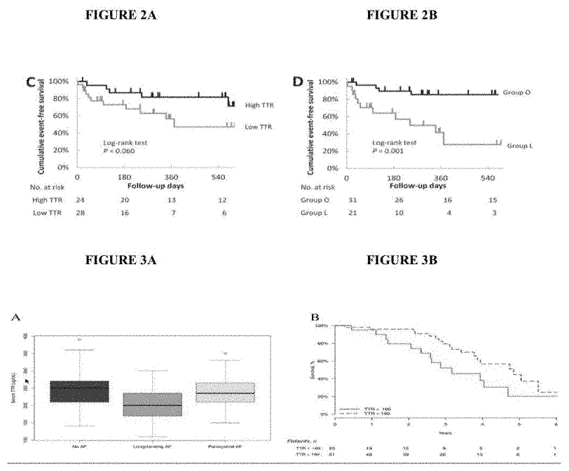

The methods provided herein also demonstrate that the risk of a poor clinical outcome increases with further reductions in TTR tetramer levels and/or functional equivalents. Statistical analyses across patients with different diseases in-fact have demonstrated every 1 mg/dL reduction in TTR tetramer levels increased the risk of death by ˜7-11%. Data comes from three independent studies by three different groups for three distinct disease processes all coming to the same conclusion that each drop in 1 mg/dL of TTR tetramer levels statistically correlates with 10% increased risk of death. The first study looked at wild type ATTR cardiomyopathy (wtATTR-CM) patients (see Hanson et al. 2018. Circ Heart Fail. 11(2):e004000). Statistically increased risk of death was observed in 101 wtATTR-CM patients if baseline TTR levels <18 mg/dL as measured by immunoturbidimetric (IT) assays (Abbot Laboratories) compared to wtATTR-CM patients with higher levels ≥18 mg/dL. The second study looked at 130 patients on hemodialysis (independent of cause of end stage renal disease requiring dialysis) (see Mittman et al., American Journal of Kidney Diseases, Volume 38, Issue 6, 1358-1364 (2001)). This study measured serum transthyretin (referred to as prealbumin) levels by rate nephelometry (Beckman Array Protein System), with baseline mean serum level of 26.5±6.5 mg/dL. This study showed patients with baseline levels of 30 mg/dL had a cumulative overall survival benefit compared to those with TTR 30 mg/dL. The third was a prospective study which looked at 52 patients diagnosed with acute heart failure (HF) (see Suzuki et al. (Int Heart J 2015; 56: 226-233). Lower event-free survival (EFS) rates were seen by Kaplan-Meier analysis in patients with baseline TTR levels <15 mg/dL compared to >15 mg/dL (HR 2.67; p=0.06. The hazard ratio increased to 4.35 (95% CI 2.01-18.62, p=0.001) if patients were stratified by both TTR levels <15 mg/dL and poor nutritional status at baseline (indicated as Group L in figure D next to C) (FIGS. 2A and 2B). We note that the third study did not specify which method was used to measure TTR levels.

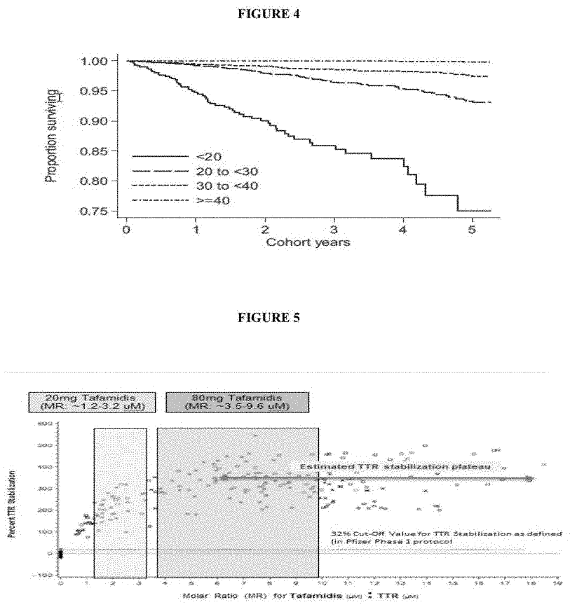

Another example comes from a 5-year study of 798 patients receiving maintenance hemodialysis with mean baseline serum TTR level of 28.3 mg/dL (median 28 mg/dL) (see Rambod, M. et al, Am J Clin Nutr 2008 88(6): 1485-94). An increased mortality rate was seen in patients with baseline TTR levels <20 mg/dL (HR 2.16, p<0.001) according to their a-priori selected prealbumin group (FIG. 4).

### Example 4

### Decreasing HMW TTR Levels Correlates with Worsening Clinical Outcomes in Patients Receiving Dialysis

In the above mentioned 5-year study of 798 patients receiving maintenance hemodialysis (Rambod 2008), it was noted that amongst the patients with baseline TTR levels between 20-40 mg/dL whose serum TTR was re-measured 6 months later, 10 mg/dL TTR level reductions resulted in a death HR of 1.45 (1.12-1.186, p=0.004). This finding highlights the importance of conducting serial TTR level measurements for gauging risk to disease and/or effectiveness of any intervention, including drug treatments and/or preventative medicine/lifestyle. We note that there are many diseases that can cause end-stage renal disease, for which dialysis is used as a treatment.

### Example 5

### Greater HMW TTR Levels Correlates with Improved Clinical

Outcomes in Patients Receiving Dialysis

In the above mentioned 5-year study of 798 patients receiving maintenance hemodialysis with overall mean baseline serum TTR level of 28.3 mg/dL (Rambod 2008), patients with 40 mg/dL had a HR of 0.61 (0.33-1.13, p=0.12). This finding albeit not statistically significant highlights the trend that higher TTR levels can be protective against poor clinical outcomes.

### Example 6

### Greater HMW TTR Levels in General Population Correlates with Improved Clinical Outcomes

In a study of 68,602 subjects from 2 prospective studies of the general population, subjects with T119M mutation (n=35) were found to have ˜17-20% higher TTR levels as measured by ELISA (ICL E-80PRE) compared to the general population (see Arterioscler Throm Vasc Biol 2013). T119M heterozygotes lived ˜5-10 years longer vs general population with a lower all-cause death-rate (p=0.04). Additionally, T119M status vs general population protected against different all vascular causes of death (p=0.04), cerebrovascular disease (p=0.008), and ischemic cerebrovascular disease (p=0.02).

### Example 7

### ATTR; Higher HMW TTR Levels Correlates with Improved Clinical Outcomes

In a study of 146 wtATTR patients, patients with a co-diagnosis of atrial fibrillation had significantly (p<0.01) lower TTR levels (mean 22.1 mg/dL) compared to wtATTR patients without a diagnosis of atrial fibrillation (mean 25.0 mg/dL). While survival was similar between these groups (p=0.46), survival differences were observed amongst the atrial fibrillation population when stratified by TTR levels >18 vs 18 mg/dL (HR 2.0, p=0.059) (Mints et al., ESC Heart Failure 2018 5(5): 772-779) (FIGS. 3A, 3B).

### Example 8

### Improving Levels of High Molecular Weight Transthyretin (TTR) Species in ATTR Patients with TTR Stabilizing Therapy Leads to Improved Clinical Outcomes

Amongst 116 wtATTR-CM patients with median TTR levels of 23 mg/dL (20-26; normal of 18-45 mg/dL), a subgroup of 35 patients were given either no treatment or off-label diflunisal, a type of NSAID with TTR tetramer stabilizing properties (see Circ Heart Fail. 2018; 11:e004000. DOI: 10.1161/CIRCHEARTFAILURE.117.004000). After one year, significantly increased TTR levels (p<0.001) after treatment with diflunisal (30 mg/dL, 25-43) vs untreated (22 mg/dL, 12-29) were significantly associated with improved one-year survival-rates vs untreated (p<0.001). TTR tetramer levels were measured by immunoturbidimetric (IT) assays by Abbot Laboratories.

### Example 9

### TTR Level Improvements Associated with Response to Anti-Tuberculosis Treatment Whereas No TTR improvement Associated with Drug Resistant TB Patients

TTR levels are also useful to evaluate treatment responsiveness to infectious diseases, including tuberculosis (TB). For example, previous studies have demonstrated utility in measuring baseline TTR levels and monitoring their change (increase, decrease, no change) to stratify patients and/or gauge treatment responsiveness for diseases that have no direct or currently known connection to TTR tetramer/HMW TTR species levels (see Luo et al., (2013) The Value of Serum Prealbumin in the Diagnosis and Therapeutic Response of Tuberculosis: A Retrospective Study. PLoS ONE 8(11): e79940):

“Furthermore, serum PA levels in most TB patients elevated slowly after using anti-TB drugs. About 9 months later, serum PA levels of 80.6% TB patients (258/320) have elevated to the normal range and the average was 194.1629.2 mg/L. However, serum PA levels of those drug-resistant TB patients sustained at a low status, and no significant increase even after 12 months. These results indicate that the change of serum PA levels are in accordance with the therapeutic effects of anti-TB drugs, which might present a good and objective marker in monitoring the treatment effects for most TB patients and warning the possibility of drug-resistance if the PA levels remain at a low state.”

This example also highlights a few additional important uses for serum TTR levels. Patients who had drug resistant tuberculosis had lower baseline TTR levels compared to healthy individuals (24 vs 13.7 mg/dL). Additionally, patients with drug resistant TB had no improvements in TTR levels after starting anti-TB drug treatment in contrast to patients who did not have drug-resistant TB and did show increased TTR levels back to “normal” ranges (˜19.4 mg/dL) after starting anti-TB therapy. This example reinforces the clinical utility of baseline TTR levels and monitoring TTR level changes as a means for gauging response to treatment and/or potential disease progression allowing for earlier intervention.

### Example 10

### Lower TTR levels in Trauma Patients Associated with Increased Death and Infectious Complications

A recent prospective observational study measured baseline TTR levels in 237 patients admitted to the ICU of a large level I trauma center. Analysis showed trauma patients with below normal TTR levels at admission (<19 mg/dL) were independently associated with higher in-hospital mortality (OR 1.2; p=0.031) and greater infectious complications (OR 1.893; p=0.028) by univariable analysis. Furthermore, of the 69 patients with subsequent TTR levels measured over 28 days or until discharge, those with higher TTR levels were independently associated with lower in-house mortality (OR 0.940; p=0.014) and fewer infectious complications (OR 0.96; p=0.008). “Transthyretin at Admission and Over Time as a Marker for Clinical Outcomes in Critically Ill Trauma Patients: A Prospective Single-Center Study,” World J Surg (2020) 44: 115. https://doi.org/10.1007/s00268-019-05140-6.

The above study is important because it provides prospective support that lower TTR levels (<19 mg/dL) were associated with poor clinical outcomes in trauma patients. Additionally, patients with higher TTR levels were associated with better clinical outcomes. References were provided therein supporting lower TTR levels as a predictor of worse outcomes in surgical, burn, critically ill patients. Related to our discovery, TTR levels were measured in many of the studies by immunoturbidity or similar techniques that primarily measure HMW TTR levels.

### Example 11

### Evaluation of TTR Stabilizer on TTR Levels in wtATTR-CM Patients

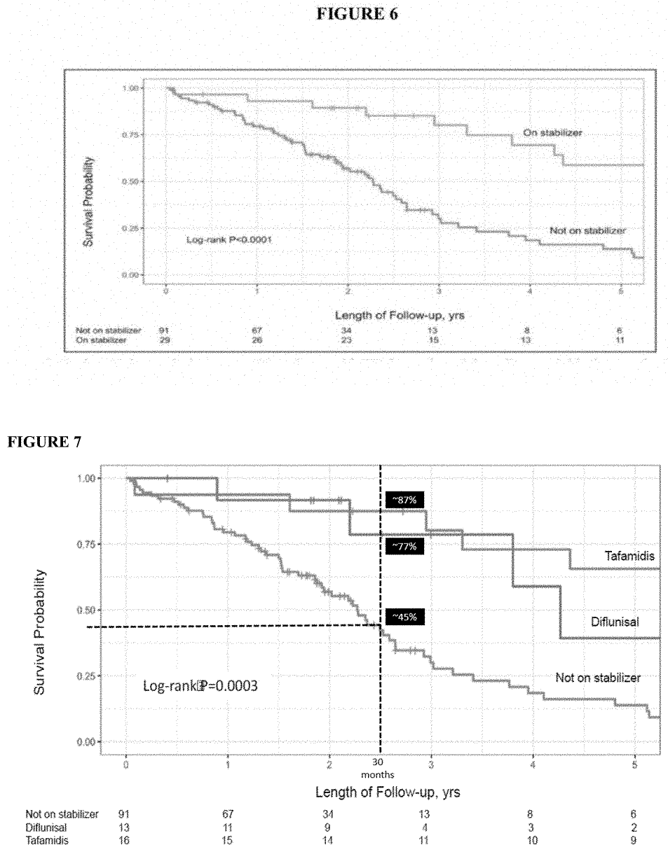

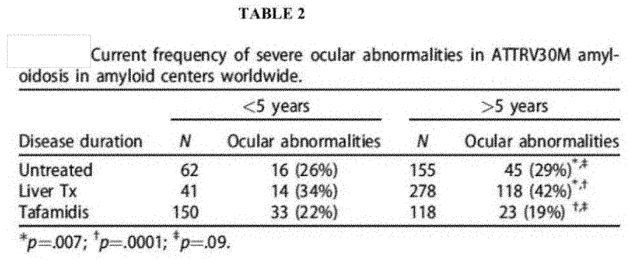

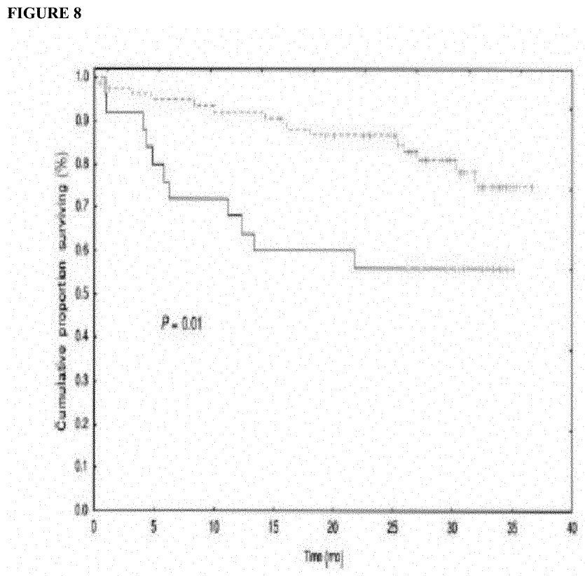

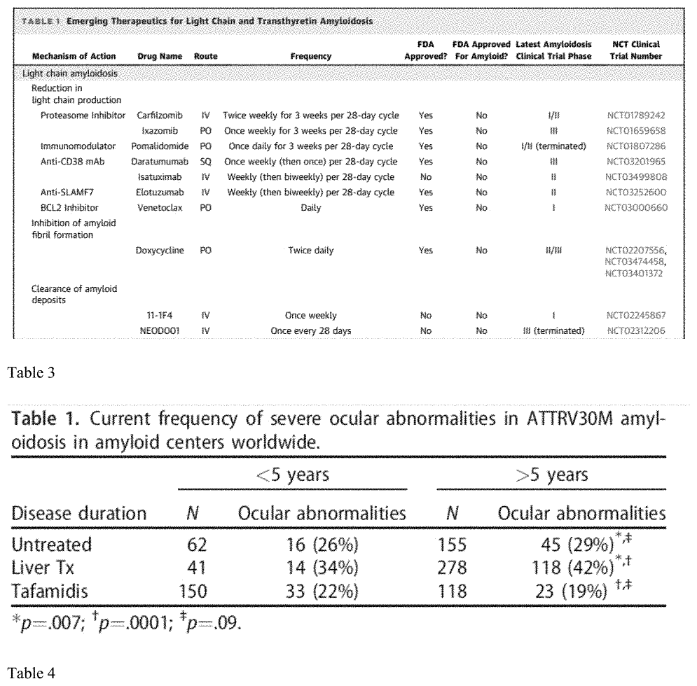

By our proprietary calculations, tafamidis 20 mg QD increased TTR levels from baseline by ˜17% in wtATTR-CM patients and ˜50-60% in V1221 mATTR-CM patients (Fx1B-201, NCT00694161) (Maurer, Mathew, et. al Effects on Transthyretin Stabilization and Clinical Outcomes. Circ Heart Fail, 2015; Blimp-Jensen, Soren. Protein Standardization. Clin Chem Lab Med 2001). We believe these levels were measured using immunoturbidimetric assays performed by Genzyme Analytic Services. Long-term follow-up of ATTR-CM patients that received 20 mg tafamidis, including at least the aforementioned four V1221 patients, showed ˜40% lower death-rates at month-30 compared to untreated (77% vs 45%). Of the 29 patients that received stabilizer treatment (tafamidis or diflunisal) vs 91 that did not, a HR of 0.32 (0.18-0.58, p<0.0001) when looking at either death or orthotopic heart transplant (OHT) (see Rosenblum, Hannah et al TTR stabilizers are associated with improved survival in patients with TTR cardiac amyloidosis. Circ Heart Fail 2018) (FIGS. 6, 7). Of note, the Phase 3 ATTR-ACT Trial (both mATTR and wtATTR-CM) showed a similar survival benefit of ˜30% reduction in death-rate at month-30 with pooled tafamidis (20 mg QD and 80 mg QD) vs placebo (see Maurer, Mathew et al. Tafamidis treatment for patients with TTR cardiomyopathy. NEJM September 2018).

Interestingly, a separate publication recently showed (1) patients who had taken oral tafamidis (presumably 20 mg QD) long-term had detectable drug levels in the vitreous fluid located in the back of the eye and 2) (see Buxbaum et al., Amyloid: The International Journal of Experimental and Clinical Investigation 2019 Jan. 24). ATTR-PN patients on long-term tafamidis treatment (>5 years) in-fact resulted in lower ocular abnormalities compared to untreated patients (p=0.09) or those who underwent liver transplantation (p=0.007), which has been the only therapeutic option for patients for many decades (Table 2). Despite prior guidance from the Company and collaborations that tafamidis did not cross the blood-brain barrier, these findings demonstrate otherwise including that tafamidis can cross the blood-brain barrier and likely accumulates there over time there. Given that TTR levels in the CSF (and likely vitreous too) are different than in the serum, we believe our discovery translating higher MW TTR isoforms and/or their equivalent could mean here that tafamidis increased localized TTR levels which resulted in improved clinical outcomes compared to other treatments (e.g., liver transplant) which may be short-lived and/or do not impact TTR levels past the blood-brain barrier.

### Example 12

### Reduced Ocular Disorders in ATTR Patients after TTR Stabilizer Therapy

A recent report highlighted increased ocular abnormalities in 804 ATTR patients with mutated TTR (V30M). Ocular abnormalities included vitreous opacities, glaucoma, abnormal conjunctival vessels, kerato-conjunctivitis sicca (KCS), scalloped pupils, retinal angiopathy, two types of familial lattice corneal dystrophy, primary gelatinous drop-like dystrophy, and dominant oculoleptomeningeal syndrome, and leptomeninges. While liver transplantation has extended the lifespan of such patients, paradoxically, it was noted that these patients had significantly increased ocular abnormalities (34%) compared to patients that only received supportive therapy (26%, no liver transplant). Moreover, this is in contrast to patients treated with tafamidis (20 mg/day dose approved ex-US for polyneuropathy) who displayed significantly fewer ocular abnormalities (22%) compared to those that received liver transplant (34%). These differences appeared to continue >5 years though statistical analyses were unable to be performed due to the group receiving tafamidis for these time points was too small to compare. “Transthyretin deposition in the eye in the era of effective therapy for hereditary ATTRV30M amyloidosis,” https://doi.org/10.1080/13506129.2018.1554563.

Multiple publications have reported reduced serum TTR levels, as measured by nephelometry or radial immunodiffusion (commercial kits readily available), in familial amyloid polyneuropathy patients with vitreous amyloidosis. “Prealbumin. A major constituent of vitreous amyloid,” Ophthalmology. 1987 July; 94(7):792-8. Given tafamidis has been shown to increase HMW TTR levels in V30M ATTR patients, decreased ocular abnormalities in tafamidis treated ATTR patients are likely due stabilized TTR, or increased HMW TTR levels. Potentially related, long-term studies have shown tafamidis was detectable within the vitreous fluid of eyes of patients taking oral tafamidis, suggesting tafamidis could have directly increased HMW TTR levels within the eye. “Transthyretin deposition in the eye in the era of effective therapy for hereditary ATTRV30M amyloidosis,” https://doi.org/10.1080/13506129.2018.1554563. See Table 4.

### Example 13

### Lower HMW TTR Levels in GI and Lung Cancer Correlate with Worse Clinical Outcomes

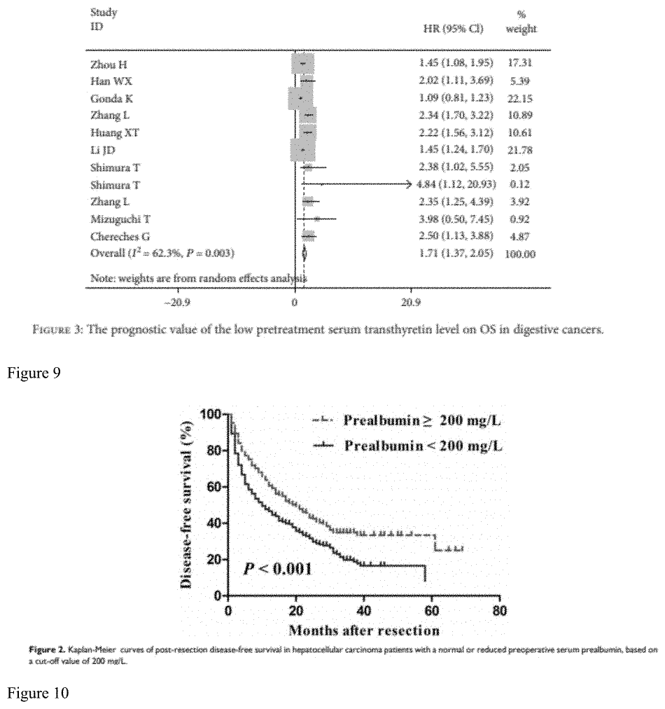

A meta-analysis of 11 studies which enrolled patients with abdominal cancers showed that those with low TTR levels had a worse overall survival (HR: 1.71; 95% CI 1.37-2.05) compared to those with normal TTR levels. The association was stronger for those with GI cancers and weaker for those with non-GI cancers (see FIG. 9; “Prognostic Value of Pretreatment Serum Transthyretin Level in Patients with Gastrointestinal Cancers,” Dis Markers. 2019 Jun. 3; 2019:7142065. doi: 10.1155/2019/7142065. eCollection 2019.).

Another study enrolled 1,349 hepatocellular carcinoma patients and showed those that underwent hepatic resection and had reduced preoperative serum TTR levels lower than 20 mg/dL were associated with lower survival compared to those that underwent surgery and had greater than or equal to 20 mg/dL TTR levels (p<0.001). “Serum Prealbumin is Negatively Associated with Survival in Hepatocellular Carcinoma Patients after Hepatic Resection,” J Cancer. 2019 Jun. 2; 10(13):3006-3011. doi: 10.7150/jca.30903. eCollection 2019. (ADVIA2400 Chemistry Liquid specific protein calibrator; Siemens, ADVIA® 2400 Chemistry System [Siemens Healthcare Diagnostics Inc]) See FIG. 10.

A prospective study that enrolled 42 patients with non-small lung cancer (NSCLC) revealed that patients with serum TTR levels less than 22 mg/dL had a lower survival (p=0.008) and recurrence-free survival (p=0.027) compared to those with serum TTR levels at least 22 mg/dL or greater (“Prognostic impact of serum transthyretin in patients with non-small cell lung cancer,” Mol Clin Oncol. 2019 June; 10(6):597-604. doi: 10.3892/mco.2019.1837. Epub 2019 Apr. 3). Beyond NSCLC, TTR levels have been reported to be a prognostic biomarker across various malignancies including esophageal (“Pre-treatment plasma proteomic markers associated with survival in esophageal cancer,” Br J Cancer. 2012 Feb. 28; 106(5):955-61. doi: 10.1038/bjc.2012.15. Epub 2012 Jan. 3), gastric (“Preoperative pre-albumin predicts prognosis of patients after gastrectomy for adenocarcinoma of esophagogastric junction,” World J Surg Oncol. 2016; 14: 279), colorectal (“Serum Albumin Is Superior to Prealbumin for Predicting Short-Term Recurrence in Patients with Operable Colorectal Cancer,” Nutrition and Cancer, 64:8, 1169-1173, DOI: 10.1080/01635581.2012.718034).

### Example 14

### Lower HMW TTR Levels Associated with Early Death in Healthy Elderly

In a populated-based prospective study of 553 men and 888 women aged 60 and older, lower levels of HMW TTR were significantly associated with increased risk of death in men (p<0.001) and women (p<0.001). Univariate analysis of men with TTR levels below 24 mg/dL for early death (less than or equal to 5 years) was significant (HR 2.42, p=0.004). Univariate analysis of women with TTR levels below 21 mg/dL for early death was also significant (HR 2.25, p=0.01). Of note, 9-year cancer mortality rate was significantly greater in men with lower HMW TTR levels. HMW TTR levels were measured using immunonephelometric methods (Immage, Beckman Coulter), which falls under methods covered by our discovery (“Biomarkers of inflammation and malnutrition associated with early death in healthy elderly people,” J Am Geriatr Soc. 2008 May; 56(5):840-6. doi: 10.1111/j.1532-5415.2008.01677.x. Epub 2008 Apr. 9). The study provides prospective clinical data to support lower HMW TTR levels are prognostic for increased risk of death in otherwise healthy elderly men and women.

### Example 15

### Alzheimer's Disease (AD); Lower HMW TTR levels in AD vs Healthy Controls

Multiple reports have studied plasma TTR levels in Alzheimer's disease (AD), which mainly affects people over the age of 65. TTR tetramer has been reported to be able to sequester beta-amyloid (Aβ) peptides (Aβ40 and Aβ42) and suppress Aβ fibrillation, a hallmark found in patients with AD. Aβ peptides are thought to be a driver of disease progression from mild cognitive impairment (MCI) to Alzheimer's disease (AD).

In a prospective study (“Biomarkers of inflammation and malnutrition associated with early death in healthy elderly people,” J Am Geriatr Soc. Author manuscript; available in PMC 2009 May 26. Published in final edited form as: J Am Geriatr Soc. 2008 May; 56(5): 840-846. Published online 2008 Apr. 9. doi: 10.1111/j.1532-5415.2008.01677.x) of 553 men and 888 women aged 60 and older, lower plasma levels of HMW TTR were significantly associated with greater risk of death in men (p<0.001) and women (p<0.001). Univariate analysis of men with TTR levels below 24 mg/dL was statistically significant (HR 2.42, p=0.004) for early death in 5 or less years. HMW TTR levels were measured by immunonephelometry (Image system, Beckman Co). In another publication (“Transthyretin decrease in plasma of MCI and AD patients: investigation of mechanisms for disease modulation,” Curr Alzheimer Res. 2012 October; 9(8):881-9), plasma TTR levels from amnesic mild cognitive impairment (aMCI) and AD patients were lower vs non-demented control patients. Serum TTR levels were measured by a radial immunodiffusion kit (Binding Site).

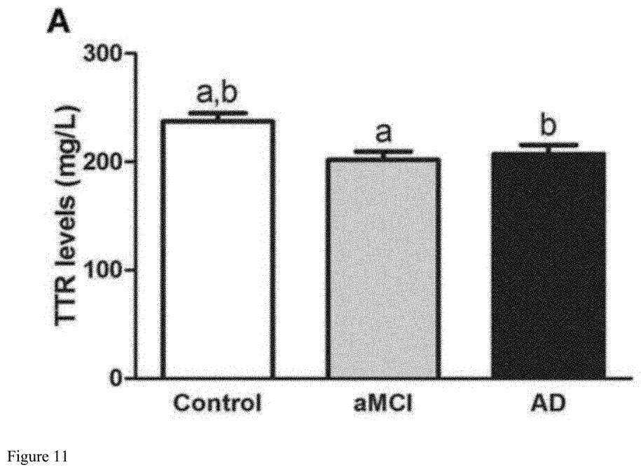

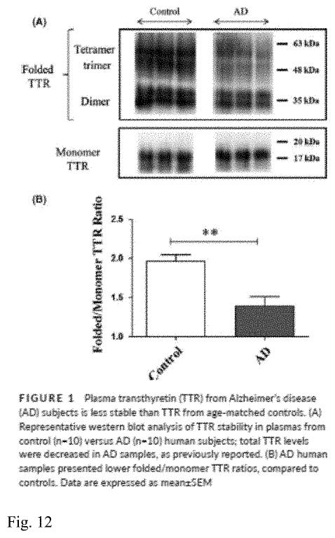

Studies have also evaluated cerebrospinal fluid (CSF) HMW TTR levels in AD, many of which reported lower CSF HMW TTR levels in AD vs healthy controls (“Reduced prealbumin (transthyretin) in CSF of severely demented patients with Alzheimer's disease,” Acta Neurol Scand. 1988 December; 78(6):455-9). For example, one study (“Cerebrospinal fluid transthyretin: aging and late-onset Alzheimer's disease,” J Neurol Neurosurg Psychiatry. 1997 October; 63(4):506-8) enrolled 149 patients, 40 of whom were elderly with dementia and probable AD according to NINCDS-ADRDA criteria. This study reported significantly lower CSF HMW TTR levels in AD patients compared to elderly controls (p<0.001). TTR levels were measured using nephelometry-based methods, which are included in our discovery as methods that yield HMW TTR levels which are clinically relevant. See FIG. 11.

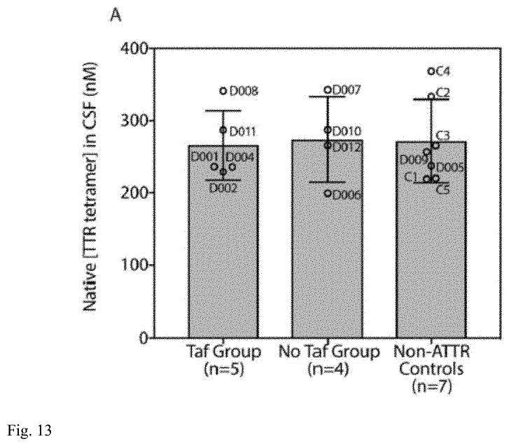

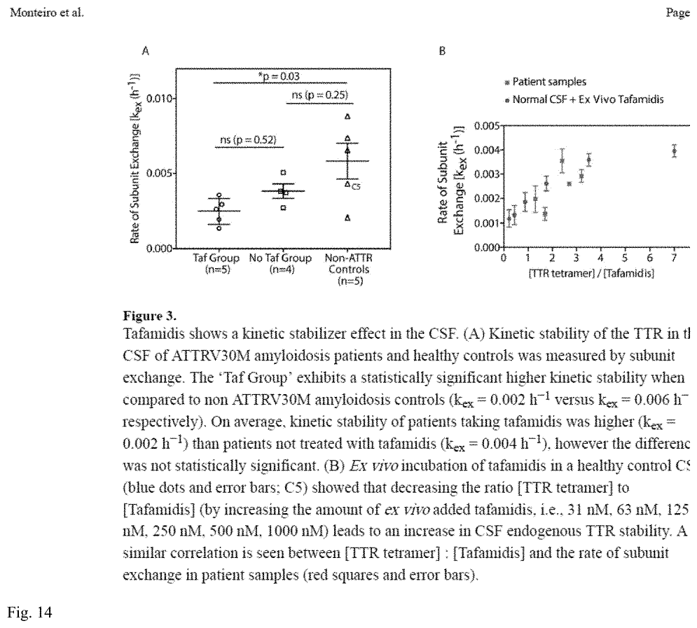

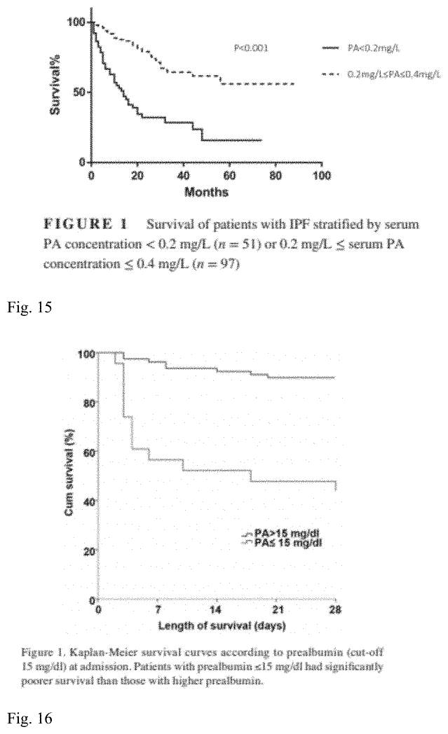

Similar conclusions have been reached by others in that higher HMW TTR levels were found in healthy controls vs AD. Higher HMW TTR levels have been presented as increased ratios of TTR folded/monomer levels in healthy controls (“Transthyretin stability is critical in assisting beta amyloid clearance- Relevance of transthyretin stabilization in Alzheimer's disease,” CNS Neurosci Ther. 2017 July; 23(7):605-619. doi: 10.1111/cns.12707. Epub 2017 Jun. 1). In AD, mutant TTR vs wild-type TTR has been reported to have decreased tetramer stability and decreased transport of beta-amyloid. These findings are important because treatment with iodo-diflunisal, a TTR tetramer stabilizing agent, demonstrated enhanced TTR-assisted beta-amyloid (Aβ) transport, a process impaired in AD. Our discovery is important and relevant because it allows for clinically functional TTR levels to be measured in bodily fluids like the CSF or plasma. See FIG. 13.

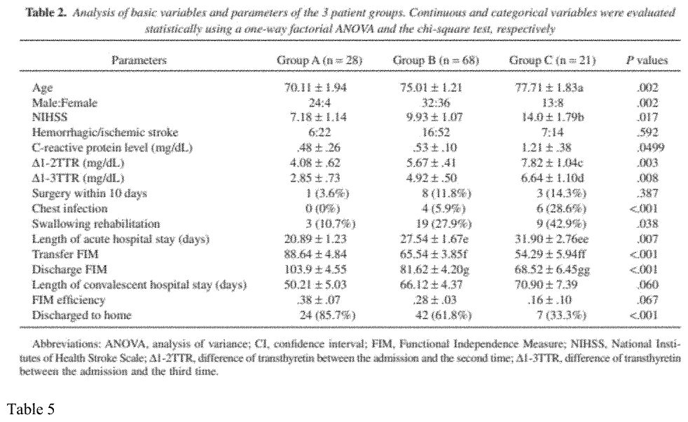

The aforementioned study used methods covered by our discovery and as such the findings of lower TTR levels in AD vs controls are clinically relevant. However, this is in contrast to another study recently published in December 2019 (“Plasma Transthyretin as a Predictor of Amnestic Mild Cognitive Impairment Conversion to Dementia,” Sci Rep. 2019 Dec. 10; 9(1):18691. doi: 10.10381s41598-019-55318-0) which used methods not covered by our discovery, which was an ELISA based technique using an anti-TTR antibody not preferential for HMW TTR species. As a result, plasma TTR levels were significantly higher in 184 patients with mild cognitive impairment (MCI) vs 40 sex- and age-matched healthy controls (36.7 vs 32.4 mg/dL, p<0.001). As the methods used here are not covered by our discovery, conclusions of higher TTR levels in early AD vs controls are not clinically relevant and should not be interpreted next to studies that used methods covered by our discovery and therefore are clinically relevant. Our discovery can be immediately adopted in the clinical arena and will help unravel existing confusion regarding the clinical reliability of TTR levels. Moreover, our discovery clarifies TTR misunderstandings and introduces a versatile clinical biomarker to patients . See FIGS. 13 and 14.

FIG. 13 shows native TTR concentration in the CSF is not significantly different between the three groups. Average [native TTR] are 265.8 nM (“Taf Group”), 273.8 nM (“no Taf Group”) and 271.5 nM (non-ATTR controls). In the “No Taf Group”, D007 and D012 are LT-treated patients, whereas D006 and D010 are untreated patients. Non-ATTR Controls includes five commercial CSFs (C1-C5). Taf: Tafamidis.

### Example 16

### Idiopathic Pulmonary Fibrosis (IPF): Greater Survival Rate if HMW TTR is 20 mg/dL or more

A study of 183 patients with idiopathic pulmonary fibrosis (IPF) revealed that those with baseline levels of at least 20 mg/dL or more had significantly greater survival rates compared to those with levels below 20 mg/dL (p<0.001). See FIG. 15. “Serum prealbumin is a prognostic indicator in idiopathic pulmonary fibrosis,” Clin Respir J. 2019 August; 13(8):493-498. doi: 10.1111/crj.13050. Epub 2019 Jun. 20.

### Example 17

### Stroke: Greater Survival Rate if HMW TTR is 15 mg/dL or More

A retrospective study of 102 heatstroke patients admitted to the ICU showed that patients with TTR levels less than or equal to 15 mg/dL had a lower survival rate compared to patients with levels greater than 15 mg/dL (p<0.001). Of note, TTR levels were reported to be measured by immunoturbidimetry, which our patent claimsa method that can measure HMW TTR species (“Effect of prealbumin level on mortality in heatstroke patients,” Experimental and Therapeutic Medicine 17: 3053-3060, 2019). See FIG. 16.

Another study investigated 117 patients with acute ischemic or hemorrhagic stroke and concluded that TTR levels of 10 mg/dL or less were predictive of poor outcomes in stroke patients undergoing convalescent rehabilitation. Interestingly, patients with an early decrease in TTR levels suggested a decreased response to rehabilitation efforts, which means TTR levels could be a useful marker to monitor in stroke patients. Patients with TTR levels greater than or equal to 20 mg/dL had significantly better functional improvement measurements compared to patients grouped with lower TTR levels. (“Transthyretin Concentrations in Acute Stroke Patients Predict Convalescent Rehabilitation,” J Stroke Cerebrovasc Dis. 2017 June; 26(6): 1375-1382. doi: 10.1016/j.jstrokecerebrovasdis.2017.02.020. Epub 2017 Mar. 14). See Table 5. Group A patients were defined as patients with high transthyretin levels (>20 mg/dL). Group C patients were defined as those with transthyretin level of lower than 10 mg/dL, which correlated with a poor outcome at 3 months. The remaining patients were defined as group B.

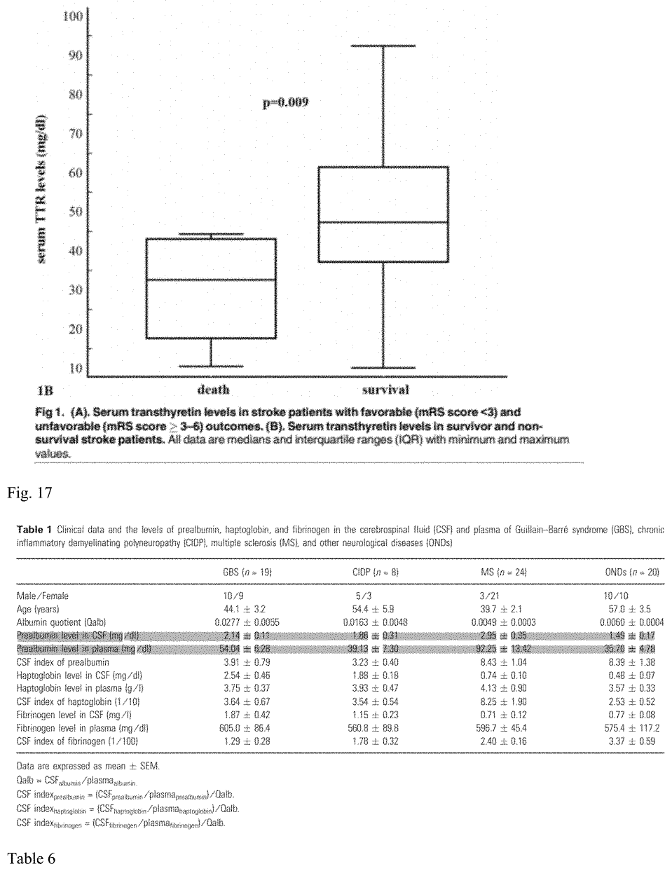

Lastly, in a study that enrolled 81 ischemic stroke patients, serum TTR levels at discharge were significantly lower in non-survivors compared to survivors (p=0.009). We note that serum TTR levels were measured by ELISA (Abeam, Cambridge, UK) in this paper, which introduces questions whether this assay is preferentially measuring concentrations of HMW TTR species. (Ambrosius W, Michalak S, Kazmierski R, Andrzejewska N, Kozubski W (2017) Predictive value of serum transthyretin for outcome in acute ischemic stroke. PLoS ONE 12(6): e0179806. https://doi.org/10.1371/journal.pone.0179806). See FIG. 17.

### Example 18

### TTR Levels are Not Negative Acute-Phase Proteins in All Inflammatory Diseases

Historically, TTR levels are perceived as always being non-specifically reduced in inflammatory states or hepatic disease. As such, TTR serum levels are thought to always behave like negative acute-phase proteins. This is not entirely accurate. A prospective study took CSF and plasma from patients with GBS (n=19), CIDP (n=8), MS (n=24), or other neurological disorders (OND, n=20). Typically, albumin CSF/plasma ratio is elevated in inflammatory neuropathies such as GBS and CIDP. This study confirmed albumin ratios were significantly greater in both GBS and CIDP when compared to OND patients. For TTR, when looking at serum and CSF TTR levels, GBS and MS patients had higher TTR levels compared to CIDP or ONDs patients. Higher TTR levels in plasma and CSF of MS patients relative to CIDP and GBS (and OND) suggests TTR levels (CSF or plasma) may not always respond as a negative acute-phase protein for all inflammatory disorders. Regardless, as TTR levels were measured by ELISA kits (Immundiagnostik AG), this raised the question whether more than HMW TTR levels were measured given ELISA values were higher than immunoturbidimetry values. “Altered cerebrospinal fluid index of prealbumin, fibrinogen, and haptoglobin in patients with Guillain-Barré syndrome and chronic inflammatory demyelinating polyneuropathy,” Acta Neurol Scand. 2012 February; 125(2):129-35. doi: 10.1111/j.1600-0404.2011.01511.x. Epub 2011 Mar 24. See Table 6.

### Example 19

### Higher HMW TTR Levels Significantly Associated with Improved Survival in Patients with ATTR-CM and Atrial Fibrillation

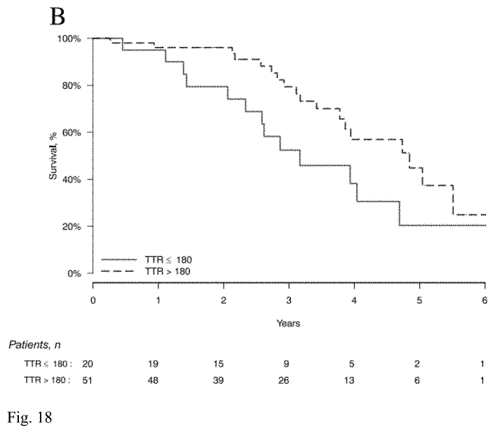

A retrospective analysis of 146 patients with biopsy-proven wtATTR-CM was conducted with results that revealed significantly lower serum TTR levels were found in those with atrial fibrillation (AF) compared to those without AF (p<0.005). Amongst patients with ATTR-CM and AF, those with TTR levels at or below 18 mg/dL had a significantly lower survival-rate compared to those with greater than 18 mg/dL (HR 2.0, p=0.059). Statistical analysis supported this survival difference was not due to differences in rhythm or rate control medications, which suggests a survival benefit could be due to higher TTR levels. Also of note, similar serum albumin, creatinine, retinol-binding protein, troponin I, and B-type natriuretic protein (BNP) was found between those with and without AF supporting differences in TTR levels were not non-specific. See FIG. 18. “Features of atrial fibrillation in wild-type transthyretin cardiac amyloidosis: a systematic review and clinical experience,” ESC Heart Failure 2018; 5: 772-779.

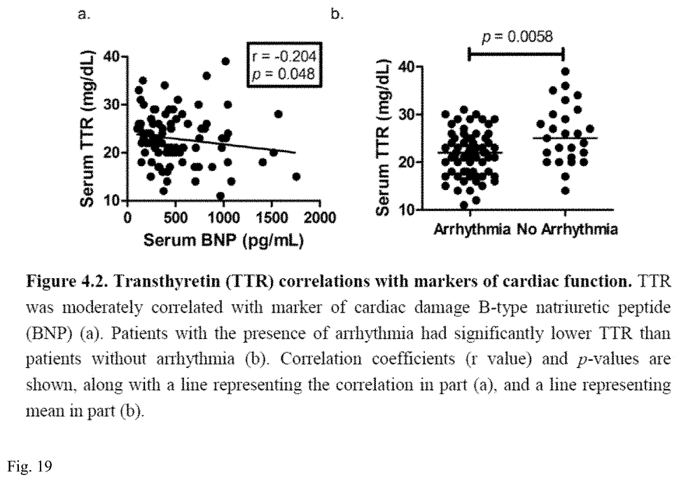

In a separate report, amongst 95 ATTRwt patients, those with arrhythmia were found to have significantly lower serum TTR levels compared to those without arrhythmia (p=0.0058). See FIG. 19. “Transthyretin gene regulation in wild-type transthyretin amyloidosis,” https://hdl.handle.net/2144/20799.

While preferred embodiments have been set forth above, those skilled in the art who have reviewed the present disclosure will readily appreciate that other embodiments can be realized within the scope of the invention. For example, features from different embodiments can be combined wherever feasible. Also, numerical quantities are illustrative rather than limiting. Further, biological samples can include blood, urine, CSF, vitreous fluid, or essentially any biological fluid where TTR levels are detectable/quantifiable via the correct method. Accordingly, the present invention should be construed as limited only by the appended claims.

What is claimed is:

1. A method involving measuring higher molecular weight (HMW) transthyretin (TTR) species, and/or functional equivalents, for identifying a human subject at risk of a poor clinical outcome and treating the subject accordingly, with the method comprising:
(a) obtaining a biological sample from the subject;
(b) measuring by use of methods including turbidity-based (i.e, immunoturbidity), nephelometry-based (i. e, immunonephelometry), single radial immunodiffusion [SRID] assays), or functional equivalents that measure HMW TTR levels thereof present in the biological sample, wherein a detected level of HMW TTR species or functional equivalents thereof below a normal threshold indicates an increased risk of a poor clinical outcome;
(c) in context of light-chain amyloidosis (AL) and associated cancer conditions, treating the patient with immunotherapy or immunomodulatory agents, wherein said treatment will increase or stabilize HMW TTR levels in patients that are treatment-responders in addition to assisting the subject's immune system in eradicating cancerous cells, with level of response depending on the organ (i.e, renal, cardiac, neurologic, hematologic) and the criteria being used;
(d) in context of amyloid-beta (Aβ) mediated diseases (i.e, ATTR-CM, ATTR-PN, bilateral carpal tunnel, etc), treating the patient while monitoring changes in higher molecular weight (HMW) transthyretin (TTR) levels or functional equivalents thereof in the biological sample, wherein the treatment will increase or stabilize HMW TTR levels in patients that are treatment-responders; and
(e) adjusting the immunotherapy or immunomodulatory agents in accordance with the changes monitored in step (c), wherein selection of agents and optimized dose-concentrations that result in longer durations of increased or stabilized HMW TTR levels correlate with improved responses and improved survival rates whereas continued declining levels suggests lack of response and earlier stopping of therapy and consideration of alternative therapies, and wherein patients are at increased risk of poor clinical outcomes and should be managed aggressively if HMW TTR levels reach or fall below 10 mg/dL.

2. The method according to claim 1, wherein each of steps (c) and (d) comprises using assays for measuring tetramer or HMW TTR function.

3. The method according to claim 1, wherein the biological sample is selected from the group consisting of a tissue sample and a bodily fluid.

4. The method according to claim 3, wherein the biological sample is selected from the group consisting of serum or plasma, urine, cerebrospinal fluid, vitreous fluid, or a combination thereof.

5. The method according to claim 1, wherein the biological sample is blood or a blood product such as serum or plasma, and wherein the threshold level for the lower limit of normal HMW TTR levels is 18-20 mg/dL.

6. The method according to claim 1, comprising detecting a level of higher molecular weight (HMW) transthyretin (TTR) species or functional equivalent thereof at multiple timepoints during the treatment regimen, and comparing the detected levels of HMW TTR species or functional equivalent thereof, wherein an increase in HMW TTR levels or functional equivalents thereof indicates that the subject is responding to the treatment, and wherein a decrease in the detected HMW TTR levels or functional equivalents thereof indicates that the subject is not or no longer responding to the current treatment regimen.

7. The method according to claim 1, comprising screening HMW TTR species or functional equivalents thereof or a combination of TTR species where TTR tetramers or functional equivalent thereof are the predominant species as a biomarker for a disease or disorder, by providing biological samples from patients at risk or suffering from the disease or disorder; detecting the level of HMW TTR levels or functional equivalent thereof in each biological sample from each patient or in the combination of TTR species in each biological sample from each patient; and correlating the detected level of TTR tetramer or functional equivalent thereof in each biological sample from each patient or in the combination of TTR species in each biological sample from each patient with disease status.

8. The method according to claim 1, comprising selecting a treatment regimen by stratifying patient population based on baseline HMW TTR levels or levels of functional equivalents thereof in a biological sample from each patient.

9. The method according to claim 1, wherein the subject is at risk of or suffering from a disease that is not currently linked to TTR or another amyloid causing precursor protein.

10. The method according to claim 1, wherein the subject has or is at risk for transthyretin-mediated amyloidosis (ATTR), including ATTR-cardiomyopathy (ATTR-CM) and ATTR-polyneuropathy (ATTR-PN).

11. The method according to claim 1 wherein the subject has or is at risk for a poor clinical outcome due to AL, protein malnutrition, stroke, coronary disease, heart failure, Alzheimer's disease, vitreous floaters, infectious disease such as tuberculosis, glaucoma, ocular myasthenia gravis, cerebral amyloid angiopathy, amyloid beta-related angiitis, atrial-fibrillation, or leptomeningeal amyloidosis and variants like oculoleptomeningeal amyloidosis.

12. The method according to claim 1, wherein step (b) comprises using an antibody or other affinity ligand that specifically binds to HMW TTR isomer (i. e, tetramer) or a methodology that preferentially detects HMW TTR species.

13. The method according to claim 1, wherein the subject is a liver transplant recipient or has received another TTR modulating agent.

14. The method according to claim 1, wherein step (b) further comprises the step of isolating TTR tetramer from other TTR isoforms in the biological sample.

15. The method according to claim 1, wherein the biological sample is a blood sample or a blood product sample, and wherein each 1 mg/dL drop below 18 to 20 mg/dL in HMW TTR levels in the blood sample or the blood product sample from that patient increases the risk of death by 7 to 11%.

16. The method according to claim 1, wherein TTR tetramer levels at or below 5 mg/dL are considered grave risks of death.

17. The method according to claim 1, wherein the lower range of normal threshold is 18-20 mg/dL.
